(19) United States
(12) Patent Application Publication
(10) Pub. No.: US 2016/0296669 A1
(43) Pub. Date: Oct. 13, 2016

(54) METHOD FOR PRODUCING GRAFT MATERIAL FOR TREATING NERVE DAMAGE

(71) Applicant: ADVANCED CENTER FOR TISSUE ENGINEERING LTD., Tokyo (JP)

(72) Inventors: Kenichi TEZUKA, Gifu (JP); Tomoko KAWAGUCHI, Gifu (JP); Takahiro KUNISADA, Gifu (JP); Toshiyuki SHIBATA, Gifu (JP); Hidefumi FUKUMITSU, Gifu (JP); Shoei FURUKAWA, Gifu (JP)

(21) Appl. No.: 14/890,569

(22) PCT Filed: May 14, 2014
(86) PCT No.: PCT/JP2014/062881

§ 371 Date: Nov. 11, 2015

(30) Foreign Application Priority Data
May 14, 2013 (JP) 2013-102426

(51) Int. Cl. A61L 27/38; C12N 5/0775; A61L 27/54

(52) U.S. Cl. CPC A61L 27/3895; A61L 27/3834; A61L 27/54; A61L 27/3878; C12N 5/0664; A61L 2430/32; A61L 2300/252; A61L 2300/64; A61L 2300/414; C12N 2501/115

(57) ABSTRACT

An object of the present invention is to provide a method for efficiently and reproducibly producing a graft material having a high recovery effect on dysfunction caused by nerve damage. The present invention provides a method for producing a graft material for treating nerve damage, including a step of culturing a dental pulp stem cell in a medium substantially containing no growth factors except FGF2, and others.

### TECHNICAL FIELD

The present invention relates to a method for producing a graft material for treating nerve damage by using a dental pulp stem cell, and others.

### BACKGROUND ART

The spinal cord is a path for transmitting kinetic and perceptual information between peripheral tissues and the brain. Injury of the spinal cord causes a severe physical disability such as motor paralysis and perceptual disorder. Neither effective treatment for this nor partial functional reconstruction is expected. In recent years, studies have been aggressively promoted worldwide; however a fundamental therapy has not yet been developed.

In this country, there are about 100,000 patients with spinal cord injury and about 5,000 patients have newly been injured per year. There are peaks of the number of patients at around 20 years old and around 60 years old. Spinal cord injury is caused by accidents during driving and sporting activities mainly in middle aged persons and caused by spinal fracture by application of minor impact in elderly persons, and others. A functional loss of a body is rarely recovered and the patients thereafter live a significantly limited life.

It has recently been reported that dysmobility in rats caused by spinal cord injury is significantly recovered when human dental pulp stem cells were grafted (Non Patent Literature 1). However, it is still desired to develop a method for treating nerve damage with higher reproducibility and higher recovery effect.

### CITATION LIST

### Non Patent Literature

Non Patent Literature 1: Sakai K. et al., J Clin Invest 122: 80-90

### SUMMARY OF INVENTION

### Technical Problem

An object of the present invention is to provide a method for efficiently and reproducibly producing a graft material having a high recovery effect on dysfunction caused by nerve damage.

### Solution to Problem

The present inventors have intensively conducted studies with a view to attaining the above object. As a result, they found that if dental pulp stem cells are cultured in a conventional medium supplemented with FGF2 at a relatively high concentration and the resultant culture is grafted to a model with spinal cord injury, the effect of recovering motor function significantly improved compared to the conventional methods; and that the recovery effect can be obtained with good reproducibility if dental pulp stem cells having a predetermined gene expression pattern are used. Based on the findings, they accomplished the present invention.

More specifically, the present invention relates to
[1] A method for producing a graft material for treating nerve damage, comprising

a step of culturing a dental pulp stem cell in a medium substantially containing no growth factors except FGF2,
[2] The method according to above [1], wherein the medium substantially containing no growth factors except FGF2 is a serum-containing base medium supplemented with FGF2 alone as a growth factor,
[3] The method according to above [2], wherein the serum in the medium has a concentration of less than 15 wt %,
[4] The method according to above [1], wherein the medium substantially containing no growth factors except FGF2 is a commercially available medium for culturing mesenchymal stem cells supplemented with FGF2 alone as a growth factor,
[5] The method according to any one of above [1] to [4], wherein FGF2 in the medium has a concentration of 5 ng/mL or more,
[6] The method according to above [5], wherein FGF2 in the medium has a concentration of 7 ng/mL or more,
[7] The method according to any one of above [1] to [6], wherein the dental pulp stem cell used is a dental pulp stem cell, in which the expression level of 10% or more of genes in the group of genes listed in Table 1 is 5 times or more as high as an average expression level of the genes in dental pulp stem cells,
[8] The method according to any one of above [1] to [7], wherein the dental pulp stem cell used is a dental pulp stem cell, in which the expression level of at least one gene selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg and Cbln2 is 5 times or more as high as an average expression level of genes in dental pulp stem cells,
[9] The method according to any one of above [1] to [8], wherein the dental pulp stem cell used is a dental pulp stem cell, in which the expression level of 10% or more of genes in the group of genes listed in Table 2 is 5 times or more as low as an average expression level of the genes in dental pulp stem cells,
[10] The method according to any one of above [1] to [9], wherein the dental pulp stem cell used is a dental pulp stem cell, in which the expression level of at least one gene selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2 and HTATSF1P2 is 5 times or more as low as an average expression level of genes in dental pulp stem cells,
[11] The method according to any one of above [1] to [6], wherein among two or more groups of dental pulp stem cells, a group of dental pulp stem cells in which the expression level of 10% or more of genes in the group of genes listed in Table 1 is 5 times or more as high as other groups of cells, is selected as the dental pulp stem cell to be used,
[12] The method according to any one of above [1] to [6] and [11], wherein among two or more groups of dental pulp stem cells, a dental pulp stem cell in which the expression level of at least one gene selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg and Cbln2 is 5 times or more as high as other groups of cells, is selected as the dental pulp stem cell to be used,
[13] The method according to any one of above [1] to [6], and [11] and [12], wherein among two or more groups of dental pulp stem cells, a dental pulp stem cell in which the expression level of 10% or more of genes in the group of genes listed in Table 2 is 5 times or more as low as other groups of cells, is selected as the dental pulp stem cell to be used,
[14] The method according to any one of above [1] to [6] and [11] to [13], wherein among two or more groups of dental pulp stem cells, a dental pulp stem cell in which the expression level of at least one gene selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2 and HTATSF1P2 is 5 times or more as low as other groups of cells, is selected as the dental pulp stem cell to be used,
[15] The method according to any one of above [1] to [14], wherein the nerve damage is spinal cord injury, cerebral infarction, intracerebral hemorrhage, subarachnoid hemorrhage, spinal hemorrhage, compression injury of nerve caused by disk herniation, sciatic nerve pain or peripheral nerve damage caused by diabetes,
[16] A graft material for treating nerve damage, comprising a dental pulp stem cell, and a medium substantially containing no growth factors except FGF2,
[17] The graft material for treating nerve damage according to above [16], wherein the medium substantially containing no growth factors except FGF2 is a serum-containing base medium supplemented with FGF2 alone as a growth factor,
[18] The graft material for treating nerve damage according to above [17], wherein the medium substantially containing no growth factors except FGF2 is a commercially available medium for culturing mesenchymal stem cells supplemented with FGF2 alone as a growth factor,
[19] The graft material for treating nerve damage according to any one of above [16] to [18], wherein the dental pulp stem cell is a dental pulp stem cell in which the expression level of 10% or more of genes in the group of genes listed in Table 1 is 5 times or more as high as an average expression level of genes of dental pulp stem cells,
[20] The graft material for treating nerve damage according to any one of above [16] to [19], wherein the dental pulp stem cell used is a dental pulp stem cell in which the expression level of at least one gene selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg and Cbln2 is 5 times or more as high as an average expression level of genes of dental pulp stem cells,
[21] The graft material for treating nerve damage according to any one of above [16] to [20], wherein the dental pulp stem cell used is a dental pulp stem cell in which the expression level of 10% or more of genes in the group of genes listed in Table 2 is 5 times or more as low as an average expression level of genes of dental pulp stem cells,
[22] The graft material for treating nerve damage according to any one of above [16] to [21], wherein the dental pulp stem cell used is a dental pulp stem cell in which the expression level of at least one gene selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2 and HTATSF1P2 is 5 times or more as low as an average of dental pulp stem cells,
[23] The graft material for treating nerve damage according to any one of above [16] to [22], wherein the nerve damage is spinal cord injury, cerebral infarction, intracerebral hemorrhage, subarachnoid hemorrhage, spinal hemorrhage, compression injury of nerve caused by disk herniation, sciatic nerve pain or peripheral nerve damage caused by diabetes,
[24] A method for treating nerve damage, comprising

a step of grafting a graft material for treating nerve damage produced by the method according any one of above [1] to [15] or the graft material for treating nerve damage according to any one of [16] to [23] to an area of nerve damage,
[25] The method according to above [24], wherein the nerve damage is spinal cord injury, cerebral infarction, intracerebral hemorrhage, subarachnoid hemorrhage, spinal hemorrhage, compression injury of nerve caused by disk herniation, sciatic nerve pain or peripheral nerve damage caused by diabetes,
[26] A kit for producing a graft material for treating nerve damage, comprising a medium and FGF2, and
[27] A method for selecting a material for a graft material for treating nerve damage from a plurality of groups of dental pulp stem cells, comprising selecting a dental pulp stem cell having at least one of the following properties (i) to (iv):

(i) the expression level of 10% or more of genes in the group of genes listed in Table 1 is 5 times or more as high as other groups of cells,

(ii) the expression level of at least one gene selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg and Cbln2 is 5 times or more as high as other groups of cells,

(iii) the expression level of 10% or more of genes in the group of genes listed in Table 2 is 5 times or more as low as other groups of cells, and

(iv) the expression level of at least one gene selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2 and HTATSF1P2 is 5 times or more as low as other groups of cells.

### Advantageous Effects of Invention

The graft material for treating nerve damage according to the present invention can be obtained in a simple method by adding FGF2 to a medium for culturing a dental pulp stem cell, and can provide a high motor function recovery effect by grafting the material.

The dental pulp stem cells, which is waste obtained from younger persons in a large amount and can be cryopreserved for a long term, are easily obtained. If the dental pulp stem cells derived from a person himself are used, a problem of immune rejection associated with grafting rarely occurs. Because dental pulp stem cells are tissue stem cells, growth of the cells is limited and thus a risk of cancerization is conceivably low compared to pluripotent stem cells.

### BRIEF DESCRIPTION OF DRAWINGS

FIG. 1 is a graph showing the measurement results of motor function recovery based on BBB score when a graft material obtained by culture in a medium substantially containing no growth factors except FGF2 (DP31F), a graft material (DP310) produced by a conventional method, and a control graft material (control) were grafted to rat models with total amputation of spinal cord.

FIG. 2 is a graph showing the measurement results of motor function recovery based on BBB score when a graft material obtained by culture in a medium substantially containing no FGF2 (DP31S), a graft material obtained by culture in a medium substantially containing no growth factors except FGF2 (DP31F) and a control graft material (control) were grafted to rat models with total amputation of spinal cord

FIG. 3 is a graph showing the measurement results of motor function recovery based on BBB score when graft materials obtained by culture in a medium substantially containing no growth factors except FGF2 (DP31F, DP74F, DP264F) to rat models with total amputation of spinal cord.

### DESCRIPTION OF EMBODIMENTS

### [Method for Producing Graft Material for Treating Nerve Damage]

An embodiment of the method for producing a graft material for treating nerve damage according to the present invention includes a step of culturing a dental pulp stem cell in a medium substantially containing no growth factors except FGF2.

In the specification, the “dental pulp stem cell” refers to a kind of tissue stem cell that can be isolated from the dental pulp. The tissue stem cell is also called as a somatic stem cell. Compared to an embryonic stem cell capable of differentiating into any types of cells, the tissue stem cell can be differentiated into limited types of cells.

The dental pulp stem cell can be collected from either one of a baby tooth and a permanent tooth and can be obtained from the dental pulp of an evulsion tooth such as a wisdom tooth and a baby tooth, which have been treated as medical waste. The dental pulp stem cell can be prepared and stored in accordance with methods known to those skilled in the art (for example, Takeda, T. et al.: J. Dent. Res., 87: 676-681, 2008; Tamaoki et al., J Dent Res. 2010 89: 773-778).

The dental pulp stem cell is a mesenchymal stem cell present in the hard tissue, similarly to the bone marrow mesenchymal stem cell, and can be subcultured in the same manner as in the bone marrow mesenchymal stem cell (for example, the method described in “Experimental Medicine, additional volume, Revised Cultured Cell Experiment Handbook, Chapter 8, Human Bone Marrow Mesenchymal Stem Cell”, published Jan. 1, 2009 (Yodosya)); however, the dental pulp stem cell has a long cellular division span and is not differentiated into a fat cell. Likewise, the dental pulp stem cell has different features from the stem cell isolated from the bone marrow.

The dental pulp stem cell is easily obtained and a culture method and a storage method for them are established, as described above. For the reasons, it has been expected to use the dental pulp stem cell as a base of a graft material for regenerative medicine. The dental pulp stem cells are collected from an evulsion tooth, after that, if necessary, proliferated by culturing to a predetermined amount. Since dental pulp stem cells can be cryopreserved for a long time, if dental pulp stem cells are isolated from many people and stored, a dental pulp stem cell bank can be formed.

The dental pulp stem cell is characterized by, for example, surface antigen STRO-1. Other than this, the dental pulp stem cell can be distinguished by a neural crest cell marker such as Nestin, SOX10 and SOX11 used as an index.

The dental pulp stem cell is known to have a high proliferation potency, compared to a bone marrow mesenchymal stem cell. It is also known that if the dental pulp stem cell is grafted together with calcium phosphate or hydroxyapatite to a mouse, dentin is formed.

In the method for producing a graft material for treating nerve damage according to the present invention, cells derived from a recipient of grafting or cells derived from a person except the recipient may be used. For example, dental pulp stem cells are isolated from e.g., a baby tooth or a wisdom tooth of a recipient of grafting, cultured and cryopreserved, and then, thawed at the time of need and used. Alternatively, a dental pulp stem cell having an identical human leukocyte antigen (HLA) with that of a recipient of grafting may be selected from the dental pulp stem cell bank and used for producing a graft material for treating nerve damage.

In the specification, the “nerve damage” refers to a damage in the central nerve and the peripheral nerve. Examples thereof include, but are not limited to, spinal cord injury, cerebral infarction, intracerebral hemorrhage, subarachnoid hemorrhage, spinal hemorrhage, compression injury of nerve caused by disk herniation, sciatic nerve pain or peripheral nerve damage caused by diabetes. The graft material of the present invention can be applied to any nerve damages as long as a therapeutic effect can be obtained by grafting. The therapeutic effect refers to an effect of curing a disease; however, the therapeutic effect is not limited to this and includes an effect of improving at least one symptom associated with a disease and an effect of inhibiting or delaying progression of a disease,and others.

In the specification, the graft recipient is not limited to humans and may include other mammals (for example, mice, rats, rabbits, dogs, cats, monkeys, sheep, cows, horses).

In the method for producing a graft material for treating nerve damage according to the present invention, a dental pulp stem cell is cultured in a medium substantially containing no growth factors except FGF2.

In the specification, the “medium substantially containing no growth factors except FGF2” means that the growth factor to be purposely added is FGF2 alone. Examples of such a medium include a serum-containing base medium supplemented with FGF2 alone as a growth factor; a serum-free base medium supplemented with FGF2 alone as a growth factor; a serum-containing base medium supplemented with FGF2 alone as a growth factor; a medium commercially available for culturing mesenchymal stem cells supplemented with FGF2 alone as a growth factor; and a medium commercially available for culturing mesenchymal stem cells supplemented with FGF2 alone as a growth factor.

In the specification, the “base medium” refers to a medium containing known low molecular-weight components alone. Non-limiting examples of the base medium known in the art include Eagle mediums such as BME (Basal medium Eagle's), MEM (Minimum essential medium) and DMEM (Dulbecco's modified Eagle's medium); RPMI (Roswell Park Memorial Institute) mediums such as RPMI1630 and RPMI1640; Fischer's medium, Ham's mediums such as F10 medium and F12 medium, MCDB mediums such as MCDB104, 107, 131, 151, 153, 170 and 202; and RITC80-7 medium. The base medium can be appropriately selected from these.

In the specification, “the serum” refers to the supernatant obtained by clotting blood, more specifically, refers to blood from which cell components and coagulation proteins are removed. The serum to be used in the present invention may be derived from any animal. Examples thereof include human serum, fetal calf serum and horse serum. When a graft material for treatment according to the present invention is grafted to a human, human serum is preferable.

Since the serum contains various growth factors, the “serum-containing base medium” basically contains such growth factors. However, a medium containing growth factors except FGF2 at the levels equivalent to those of the serum is defined to be the “medium substantially containing no growth factors except FGF2” in the specification. The concentration of the serum in the medium is preferably e.g., less than 15 wt %, less than 13 wt %, less than 10 wt %, less than 8 wt % or less than 5 wt %.

In the specification, the “growth factor” refers to any type of protein called a growth factor or a proliferative factor. Examples thereof include epidermal growth factor (EGF), fibroblast growth factor (FGF), acid fibroblast growth factor (aFGF or FGF1), basic fibroblast growth factor (bFGF or FGF2), platelet-derived growth factor (PDGF), nerve growth factor (NGF), insulin-like growth factor (IGF), hepatocyte growth factor (HGF), transforming growth factor (TGF), vascular endothelial growth factor (VEGF) and keratinocyte growth factor (KGF) interleukins, and others.

In the specification, the “medium commercially available for culturing mesenchymal stem cells” refers to a commercially available medium for culturing and proliferating mesenchymal stem cells while maintaining a differentiation potency and not inducing differentiation. Examples thereof include, but are not limited to, MSCGM medium (LONZA), mesenchymal stem cell growth medium (Takara Bio Inc.), mesenchymal stem cell growth medium DXF (Takara Bio Inc.), StemLine (registered trade mark) mesenchymal stem cell growth medium (Sigma-Aldrich), MF-medium (trade mark) mesenchymal stem cell growth medium (Toyobo Life Science), BD Mosaic (trade mark) and a serum-free culture kit for human mesenchymal stem cells (BD BIOSCIENCES). Since some of these commercially available mediums contain secret components and low-level serum, mediums occasionally contain various growth factors; however, as long as the growth factor to be purposely added to these mediums is FGF2 alone, these mediums correspond to the “medium substantially containing no growth factors except FGF2” of the present invention. In this case, the level of the serum is preferably less than 15%, less than 13%, less than 10%, less than 8% or less than 5%.

In the specification, FGF2 refers to a basic fibroblast growth factor (FGF) and also referred to as bFGF or HBGF-2.

FGF2 used herein can be prepared by appropriately diluting a commercially available FGF2. Since FGF2 is to be used in a graft material, FGF2 is filtered by an appropriate membrane and preferably confirmed to be negative to e.g., bacteria, fungi and mycoplasma. The concentration of FGF2 is not particularly limited as long as the resultant graft material has a sufficient spinal cord injury therapeutic effect; however, the concentration can be specified as, for example, 5 ng/mL or more or 7 ng/mL or more.

In the specification, the “medium supplemented with FGF2 alone as a growth factor” may contain e.g., other proteins as long as a growth factor except FGF2 is not added.

Examples of the substance to be added to the medium include hormones such as insulin, glucagon, prolactin, thyroxine, growth hormone, follicle stimulating hormone (FSH), luteinizing hormone (LH), thyroid hormone, estradiol and glucocorticoid; binding proteins such as ceruloplasmin, transferrin and lipoprotein; cell adhesion factors such as collagen, fibronectin, laminin and vitronectin; lipids such as prostaglandins, phospholipids and unsaturated fatty acids; and various low molecular-weight compounds. These can be used singly or in arbitrary combination. The concentrations of these substances may be appropriately selected by those skilled in the art.

To the medium to be used in the method for producing a graft material for treating spinal cord injury according to the present invention, other substances useful for culturing cells can be appropriately added. Examples of the substances include, but are not limited to, a buffer for stabilizing pH (e.g., HEPES), phenol red serving as a pH indicator, antibiotic substances (e.g., penicillin G, streptomycin, amphotericin B, gentamicin, kanamycin, ampicillin, minocycline, gentashin), amino acids, vitamins, lipids, carbohydrates, nucleic acids, inorganic salts, organic acid salts and minerals, and others.

The medium to be used in the method for producing a graft material for treating spinal cord injury according to the present invention can be prepared by dissolving requisite components in water, a buffer or a commercially available medium.

As the water to be used for preparing the medium, ultra-pure water compatible to pure water for injection is desirably used.

The medium is also aseptically prepared in a high-standard clean room or a clean bench and dispensed through a sterile filter having a pore size of 0.1 μm or less and capable of removing mycoplasma.

The storage container of the medium is preferably a plastic container made of e.g., a poly (ethylene terephthalate) co-polymer rather than a glass container, since proteins are likely to adsorb to the inner wall of the glass container.

The medium prepared may be subjected to various quality evaluation tests (such as physical property tests including measurement of e.g., pH and osmotic pressure; microorganism tests for examining contamination with e.g., bacteria, fungi and mycoplasma; virus tests for examining contamination with e.g., hepatitis virus and HIV; measurement for endotoxin level; and tests for biological activities such as cell proliferation and physiological function).

In the method for producing a graft material for treating nerve damage according to the present invention, it is preferable that a dental pulp stem cell is also subcultured in the aforementioned medium, twice, 3 times, 4 times, 5 times or 6 times or more.

The culture method is not particularly limited as long as culture is carried out in a medium substantially containing no growth factors except FGF2. Various conditions (such as temperature, humidity, CO₂ concentration, pH, frequency of exchanging medium) can be selected by those skilled in the art depending on the type of cell to be cultured.

The culture period can be appropriately determined by those skilled in the art depending upon the type of cell and the composition of the medium. For example, whether cells reach the state suitable for grafting may be determined based on the shape of the cells and the proliferation rate thereof by those skilled in the art. As the state suitable for grafting, for example, the state where the shape of a cell changed into a thin and long shape and the state where cell proliferation speed decreases may be mentioned, but not limited to these states.

In the method for producing a graft material for treating nerve damage according to the present invention, cells may be cultured by any method such as a single layer stationary culture, a rotary culture, a microcarrier culture, a suspension culture, a gyratory culture, a spheroid culture, a culture within gel and a culture by a three-dimensional carrier.

The single layer stationary culture is a method of culturing cells of a single layer by attaching the cells on the wall of a culture container. A glass or plastic culture container may be used. As the plastic, a plastic whose surface has been treated to be appropriately hydrophilic, can be used. Depending upon the type of cell and the purpose of an experiment, the plastic may be coated with an extracellular matrix such as collagen, gelatin, laminin, fibronectin and matrigel. As the coating material, collagen crosslinked by UV irradiation and gelatin obtained by treating collagen with heat can be used.

The rotatory culture is a culture method by placing a culture container in a rotatory metal drum. Large scale culture can be made if e.g., a bottle type culture container is used.

The microcarrier culture is a culture method using carriers like beads. More specifically, cells are allowed to adhere to the surface of the beads and culture is made by stirring a medium containing the beads and suspending them. This method is suitable for large-scale culture.

The suspension culture is a method of culturing cells while suspending the cells in a medium. Adhesive cells may be forcibly suspended by stirring the medium and cultured. A large amount of cells can be collected compared to the single layer culture.

The gyratory culture refers to a culture method by horizontally rotating a culture container. This is used as one of the suspension cultures and also used for forming spheroids taking advantage of a nature: suspended solids assemble to the center by gyration.

The spheroid culture is a method for forming spheroids through mutual adhesion of cells by suspending cells such that the cells are in loose contact with each other. Many of the cells obtained by the spheroid culture highly express function.

The culture within gel is a method of culturing cells by embedding the cells within e.g., collagen gel, soft agar or synthetic polymer gel and suitable for three-dimensional culture.

The three-dimensional carrier culture is a culture method using a carrier so as to three-dimensionally proliferating cells at a high density in order to enhance expression of function of cultured cells. As the carrier, a porous polymer and beads are generally used. To facilitate nutrition and gas exchange of cells densely present, a circulation system by a bioreactor is employed.

The method for producing a graft material for treating nerve damage according to the present invention may include, in addition to the aforementioned culture step, various steps appropriate for producing a graft material. For example, a step of controlling flowability of the culture obtained in the culture step by mixing the culture with a highly viscose substance such as hyaluronic acid, collagen gel, fibrinogen, soft agar and a synthetic polymer, may be carried out. By appropriately controlling flowability, a graft material can be settled at a damage site.

After mixing with gel such as collagen gel, soft agar or a synthetic polymer, culture is performed in a certain period of time and then a three-dimensional culture may be performed.

The dental pulp stem cell to be used for producing a graft material for treating nerve damage according to the specification may be a dental pulp stem cell in which the expression level of the group of genes listed in Table 1 is high compared to the average expression level of genes in dental pulp stem cells.

In the specification, the “dental pulp stem cell, in which the expression level of the group of genes listed in Table 1 is high compared to the average expression level of genes in dental pulp stem cells” refers to a dental pulp stem cell satisfying the following condition: when gene expression pattern was checked with respect to expression of the group of genes described in Table 1, the expression levels of 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 98% or more of genes, each are 5 times or more, 10 times or more, 20 times or more, 30 times or more, 40 times or more, 50 times or more, 60 times or more, 70 times or more, 80 times or more, 90 times or more, 95 times or more, or 100 times or more as high as the average in dental pulp stem cells.

In the specification, the “average expression level of genes in dental pulp stem cells” refers to an average of expression levels of genes in an arbitrary number (two or more) of dental pulp stem cells.

TABLE 1

| | | Times |
|---|---|---|
| GeneSymbol | Explanation [Genbank Accession Number] | (DP31/DP264) |
| ABCA6 | Homo sapiens ATP-binding cassette, sub-family A (ABC1), member 6 (ABCA6), mRNA [NM_080284] | 5.0137186 |
| ACVR1C | Homo sapiens activin A receptor, type IC (ACVR1C), transcript variant 1, mRNA [NM_145259] | 13.918116 |
| ADAM20 | Homo sapiens ADAM metallopeptidase domain 20 (ADAM20), mRNA [NM_003814] | 66.42648 |
| ADAMTS19 | Homo sapiens ADAM metallopeptidase with thrombospondin type 1 motif, 19 (ADAMTS19), mRNA | 8.167267 |
| | [NM_133638] | |
| ADORA1 | Homo sapiens adenosine A1 receptor (ADORA1), transcript variant 1, mRNA [NM_000674] | 6.0168715 |
| AGTR1 | Homo sapiens mRNA for angiotensin II type 1b receptor, complete cds, [D13814] | 41.855778 |
| ALDH3A1 | Homo sapiens aldehyde dehydrogenase 3 family, member A1 (ALDH3A1), transcript variant 2, mRNA | 9.322075 |
| | [NM_000691] | |
| ALDH5A1 | Homo sapiens aldehyde dehydrogenase 5 family, member A1 (ALDH5A1), nuclear gene encoding | 7.077404 |
| | mitochondrial protein, transcript variant 1, mRNA [NM_170740] | |
| ANKRD30A | Homo sapiens ankyrin repeat domain 30A (ANKRD30A), mRNA [NM_052997] | 11.803563 |
| ANXA8L2 | annexin A8-like 2 [Source: HGNC Symbol; Acc: 23335] [ENST00000340243] | 9.319177 |
| APBB1IP | Homo sapiens amyloid beta (A4) precursor protein-binding, family B, member 1 interacting protein | 6.3065367 |
| | (APBB1IP), mRNA [NM_019043] | |
| APBB1IP | Homo sapiens amyloid beta (A4) precursor protein-binding, family B, member 1 interacting protein | 6.6869807 |
| | (APBB1IP), mRNA [NM_019043] | |
| APBB1IP | Homo sapiens amyloid beta (A4) precursor protein-binding, family B, member 1 interacting protein | 20.242624 |
| | (APBB1IP), mRNA [NM_019043] | |
| APOA4 | Homo sapiens apolipoprotein A-IV (APOA4), mRNA [NM_000482] | 11.783757 |
| APOBR | Homo sapiens apolipoprotein B receptor (APOBR), mRNA [NM_018690] | 26.163698 |
| AREG | Homo sapiens amphiregulin (AREG), mRNA [NM_001657] | 6.356535 |
| ARHGAP20 | Homo sapiens Rho GTPase activating protein 20 (ARHGAP20), mRNA [NM_020809] | 8.251349 |
| ARHGAP28 | Homo sapiens Rho GTPase activating protein 28 (ARHGAP28), mRNA [NM_001010000] | 5.581529 |
| ASZ1 | Homo sapiens ankyrin repeat, SAM and basic leucine zipper domain containing 1 (ASZ1), transcript variant | 5.8410993 |
| | 1, mRNA [NM_130768] | |
| ATG9B | Homo sapiens ATG9 autophagy related 9 homolog B (S. cerevisiae) (ATG9B), mRNA [NM_173681] | 5.327448 |
| ATP6V1B1 | Homo sapiens ATPase, H+ transporting, lysosomal 56/58 kDa, V1 subunit B1 (ATP6V1B1), mRNA | 13.998029 |
| | [NM_001692] | |
| ATPBD4 | Homo sapiens ATP binding domain 4 (ATPBD4), transcript variant 1, mRNA [NM_080650] | 5.216696 |
| AVPR1A | Homo sapiens arginine vasopressin receptor 1A (AVPR1A), mRNA [NM_000706] | 5.8251414 |
| AVPR2 | Homo sapiens arginine vasopressin receptor 2 (AVPR2), transcript variant 1, mRNA [NM_000054] | 5.093321 |
| BANF2 | Homo sapiens barrier to autointegration factor 2 (BANF2), transcript variant 2, mRNA [NM_001014977] | 6.488925 |
| BATE | Homo sapiens basic leucine zipper transcription factor, ATF-like (BATE), mRNA [NM_006399] | 9.837393 |
| BHLHE22 | Homo sapiens basic helix-loop-helix family, member e22 (BHLHE22), mRNA [NM_152414] | 13.069403 |
| C10orf82 | Homo sapiens chromosome 10 open reading frame 82 (C10orf82), mRNA [NM_144661] | 12.051886 |
| C11orf96 | Homo sapiens chromosome 11 open reading frame 96 (C11orf96), mRNA [NM_001145033] | 7.144934 |
| C12orf53 | Homo sapiens chromosome 12 open reading frame 53 (C12orf53), transcript variant 1, mRNA [NM_153685] | 50.230335 |
| C14orf129 | Homo sapiens chromosome 14 open reading frame 129 (C14orf129), mRNA [NM_016472] | 5.886228 |
| C17orf81 | Homo sapiens chromosome 17 open reading frame 81 (C17orF81), transcript variant 3, mRNA | 6.2237744 |
| | [NM_203414] | |
| C1orf162 | Homo sapiens chromosome 1 open reading frame 162 (C1orf162), mRNA [NM_174896] | 9.743706 |
| C1orf226 | Homo sapiens chromosome 1 open reading frame 226 (C1orf226), transcript variant 2, mRNA | 6.454425 |
| | [NM_001085375] | |
| C1orf64 | Homo sapiens chromosome 1 open reading frame 64 (C1orF64), mRNA [NM_178840] | 45.61271 |
| C1orf81 | Homo sapiens C1orf81 mRNA, partial seauence, [DQ983818] | 8.118939 |
| C20orf103 | Homo sapiens chromosome 20 open reading frame 103 (C20orf103), transcript variant 1, mRNA | 7.7917347 |
| | [NM_012261] | |
| C20orf201 | Homo sapiens chromosome 20 open reading frame 201 (C20orf201), mRNA [NM_001007125] | 8.537778 |
| C2orf48 | Homo sapiens chromosome 2 open reading frame 48 (C2orf48), mRNA [NM_182626] | 42.43253 |
| C6orf124 | Homo sapiens chromosome 6 open reading frame 124 (C6orf124), non-coding RNA [NR_027906] | 12.911 |
| C9orf47 | Homo sapiens cDNA FLJ37523 fis, clone BRCAN2006401, [AK094842] | 5.6207094 |
| CACNG6 | Homo sapiens calcium channel, voltage-dependent, gamma subunit 6 (CACNG6), transcript variant 1, | 8.660492 |
| | mRNA [NM_145814] | |
| CACNG7 | Homo sapiens calcium channel, voltage-dependent, gamma subunit 7 (CACNG7), mRNA [NM_031896] | 16.299984 |
| CAMTA1 | Homo sapiens calmodulin binding transcription activator 1 (CAMTA1), transcript variant 1, mRNA | 8.311245 |
| | [NM_015215] | |
| CAPN13 | Homo sapiens calpain 13 (CAPN13), mRNA [NM_144575] | 6.583616 |
| CARD14 | Homo sapiens caspase recruitment domain family, member 14 (CARD14), transcript variant 1, mRNA | 10.370044 |
| | [NM_024110] | |
| CARD6 | Homo sapiens caspase recruitment domain family, member 6 (CARD6), mRNA [NM_032587] | 7.9052444 |
| CBLN2 | Homo sapiens cerebellin 2 precursor (CBLN2), mRNA [NM_182511] | 235.30481 |
| CCDC144A | Homo sapiens coiled-coil domain containing 144A (CCDC144A), mRNA [NM_014695] | 7.993268 |
| CCDC144A | Homo sapiens coiled-coil domain containing 144A (CCDC144A), mRNA [NM_014695] | 14.71254 |
| CCDC144NL | Homo sapiens coiled-coil domain containing 144 family, N-terminal like (CCDC144NL) mRNA | 7.063268 |
| | [NM_001004306] | |
| CCL2 | Homo sapiens chemokine (C-C motif) ligand 2 (CCL2), mRNA [NM_002982] | 14.825152 |
| CCND2 | Homo sapiens cyclin D2 (CCND2), mRNA [NM_001759] | 5.8978915 |
| CCRL1 | Homo sapiens chemokine (C-C motif) receptor-like 1 (CCRL1), transcript variant 1, mRNA [NM_178445] | 14.8459 |
| CD1D | Homo sapiens CD1d molecule (CD1D), mRNA [NM_001766] | 5.5443244 |
| CDC20B | cell division cycle 20 homolog B (S. cerevisiae) [Source: HGNC Symbol; Acc:24222] [ENST00000507931] | 32.347153 |
| CDCP1 | Homo sapiens CUB domain containing protein 1 (CDCP1), transcript variant 2, mRNA [NM_178181] | 5.723103 |
| CDH6 | cadherin 6 type 2, K-cadherin (fetal kidney) [Source: HGNC Symbol; Acc: 1765] [ENST00000506396] | 5.805565 |
| CDR1 | Homo sapiens cerebellar degeneration-related protein 1, 34 kDa (CDR1), mRNA [NM_004065] | 7.8617926 |
| CEBPA | Homo sapiens CCAAT/enhancer binding protein (C/EBP), alpha (CEBPA), mRNA [NM_004364] | 5.1645403 |
| CFH | Homo sapiens complement factor H (CFH), nuclear gene encoding mitochondrial protein, transcript variant | 7.511274 |
| | 1, mRNA [NM_000186] | |
| CFH | Homo sapiens complement factor H (CFH) nuclear gene encoding mitochondrial protein, transcript variant 1, | 9.279625 |
| | mRNA [NM_000186] | |
| CFHR3 | Homo sapiens complement factor H-related 3 (CFHR3) transcript variant 1, mRNA [NM_021023] | 6.1217637 |
| CFI | Homo sapiens complement factor I (CFI), mRNA [NM_000204] | 5.176732 |
| CFI | Homo sapiens complement factor I (CFI), mRNA [NM_000204] | 6.772929 |
| CFTR | Homo sapiens cystic fibrosis transmembrane conductance regulator (ATP-binding cassette sub-family C, | 11.484309 |
| | member 7) (CFTR), mRNA [NM_000492] | |
| CH25H | Homo sapiens cholesterol 25-hydroxylase (CH25H), mRNA [NM_003956] | 18.601637 |
| CHRDL2 | Homo sapiens chordin-like 2 (CHRDL2), mRNA [NM_015424] | 6.799207 |
| CHRM2 | Homo sapiens cholinergic receptor, muscarinic 2 (CHRM2), transcript variant 2, mRNA [NM_001006627] | 5.7264347 |
| CLEC4C | Homo sapiens C-type lectin domain family 4 member C (CLEC4C) transcript variant 1, mRNA | 7.951971 |
| | [NM_130441] | |
| CMTM8 | Homo sapiens CKLF-like MARVEL transmembrane domain containing 8 (CMTM8), mRNA [NM_178868] | 6.18745 |
| CNGA3 | Homo sapiens cyclic nucleotide gated channel alpha 3 (CNGA3), transcript variant 1, mRNA [NM_001298] | 18.462748 |
| CNTN6 | Homo sapiens contactin 6 (CNTN6), mRNA [NM_014461] | 9.551367 |
| CNTNAP3B | contactin associated protein-like 3B [Source: HGNC Symbol; Acc: 32035] [ENST00000276974] | 6.5607357 |
| COL3A1 | Homo sapiens collagen, type III, alpha 1 (COL3A1), mRNA [NM_000090] | 5.7362475 |
| COL3A1 | Homo sapiens collagen, type III, alpha 1 (COL3A1), mRNA [NM_000090] | 10.846549 |
| COL6A5 | Homo sapiens collagen, type VI, alpha 5 (COL6A5), mRNA [NM_153264] | 26.53334 |
| CPXM2 | Homo sapiens carboxypeptidase X (M14 family), member 2 (CPXM2), mRNA [NM_198148] | 5.008564 |
| CPZ | Homo sapiens carboxypeptidase Z (CPZ), transcript variant 3, mRNA [NM_001014448] | 5.3078647 |
| CREG2 | Homo sapiens cellular repressor of E1A-stimulated genes 2 (CREG2) mRNA [NM_153836] | 5.011623 |
| CSN2 | Homo sapiens casein beta (CSN2), mRNA [NM_001891] | 77.05627 |
| CTAG1A | Homo sapiens cancer/testis antigen 1A (CTAG1A), mRNA [NM_139250] | 88.44661 |
| CTAG2 | Homo sapiens cancer/testis antigen 2 (CTAG2), transcript variant 2, mRNA [NM_020994] | 20.843609 |
| CXCL1 | Homo sapiens chemokine (C-X-C motif) ligand 1 (melanoma growth stimulating activity, alpha) (CXCL1), | 13.791409 |
| | mRNA [NM_001511] | |
| CXCL2 | Homo sapiens chemokine (C-X-C motif) ligand 2 (CXCL2), mRNA [NM_002089] | 8.983284 |
| CXCL6 | Homo sapiens chemokine (C-X-C motif) ligand 6 (granulocyte chemotactic protein 2) (CXCL6), mRNA | 5.8617096 |
| | [NM_002993] | |
| CXCR3 | Homo sapiens chemokine (C-X-C motif) receptor 3 (CXCR3), transcript variant 1, mRNA [NM_001504] | 28.491007 |
| CYP26B1 | Homo sapiens cytochrome P450, family 26, subfamily B, polypeptide 1 (CYP26B1), mRNA [NM_019885] | 8.024849 |
| DCLK1 | Homo sapiens doublecortin-like kinase 1 (DCLK1), transcript variant 1, mRNA [NM_004734] | 24.201939 |
| DCT | Homo sapiens mRNA for tyrosinase related protein-2 partial, axons 7, 8, 8b (alternative) and 3′UTR, | 53.528954 |
| | [AJ132932] | |
| DCTN1 | dynactin 1 [Source: HGNC Symbol; Acc: 2711] [ENST00000462813] | 14.119596 |
| DIRAS3 | Homo sapiens DIRAS family, GTP-binding RAS-like 3 (DIRAS3), mRNA [NM_004675] | 7.6576667 |
| DLEU7 | deleted in lymphocytic leukemia, 7 [Source: HGNC Symbol; Acc: 17567] [ENST00000504404] | 18.614939 |
| DLX6 | Homo sapiens distal-less homeobox 6 (DLX6), mRNA [NM_005222] | 5.0957623 |
| DNAH1 | Homo sapiens dynein, axonemal, heavy chain 1 (DNAH1), mRNA [NM_015512] | 85.367615 |
| DPP6 | Homo sapiens dipeptidyl-peptidase 6 (DPP6), transcript variant 3, mRNA [NM_001039350] | 5.389067 |
| DTX4 | Homo sapiens dettex homolog 4 (Drosophila) (DTX4), mRNA [NM_015177] | 14.33478 |
| DYDC2 | Homo sapiens DPY30 domain containing 2 (DYDC2), mRNA [NM_032372] | 50.603565 |
| ECE2 | Homo sapiens endothelin converting enzyme 2 (ECE2), transcript variant 1, mRNA [NM_014693] | 5.7223144 |
| EDNRB | Homo sapiens endothelin receptor type B (EDNRB), transcript variant 2, mRNA [NM_003991] | 5.2736936 |
| EDNRB | Homo sapiens endothelin receptor type B (EDNRB), transcript variant 2, mRNA [NM_003991] | 6.9502497 |
| EGR3 | Homo sapiens early growth response 3 (EGR3), transcript variant 1, mRNA [NM_004430] | 5.7416267 |
| ELN | Homo sapiens elastin (ELN), transcript variant 1, mRNA [NM_000501] | 15.548598 |
| EMR1 | Homo sapiens egf-like module containing, mucin-like, hormone receptor-like 1 (EMR1), mRNA | 8.012805 |
| | [NM_001974] | |
| ENTPD1 | Homo sapiens ectonucleoside triphosphate diphosphohydrolase 1 (ENTPD1), transcript variant 1, mRNA | 11.598013 |
| | [NM_001776] | |
| ENTPD1 | Homo sapiens ectonucleoside triphosphate diphosphohydrolase 1 (ENTPD1), transcript variant 1 mRNA | 18.57545 |
| | [NM_001776] | |
| ENTPD3 | Homo sapiens ectonucleoside triphosphate diphosphohydrolase 3 (ENTPD3), mRNA [NM_001248] | 6.0331593 |
| EPHA6 | Homo sapiens EPH receptor A6 (EPHA6), transcript variant 2, mRNA [NM_173655] | 7.9318533 |
| ERG | Homo sapiens v-ets erythroblastosis virus E26 oncogene homolog (avian) (ERG), transcript variant 2, mRNA | 6.8761225 |
| | [NM_004449] | |
| ETV1 | Homo sapiens ets variant 1 (ETV1), transcript variant 1, mRNA [NM_004956] | 5.930012 |
| ETV1 | Homo sapiens ets variant 1 (ETV1) transcript variant 1, mRNA [NM_004956] | 6.6959066 |
| EYA4 | Homo sapiens eves absent homolog 4 (Drosophila) (EYA4), transcript variant 1, mRNA [NM_004100] | 5.8544316 |
| FAM104B | Homo sapiens family with sequence similarity 104, member B (FAM104B), transcript variant 1, mRNA | 7.5034156 |
| | [NM_138362] | |
| FAM106A | Homo sapiens family with sequence similarity 106, member A (FAM106A) non-coding RNA [NR_026809] | 5.367256 |
| FAM150A | Homo sapiens family with sequence similarity 150, member A (FAM150A), mRNA [NM_207413] | 6.2002835 |
| FAM5C | Homo sapiens family with sequence similarity 5, member C (FAM5C), mRNA [NM_199051] | 6.07245 |
| FAM65C | Homo sapiens family with sequence similarity 65, member C (FAM65C), mRNA [NM_080829] | 6.6242995 |
| FAM81A | Homo sapiens family with sequence similarity 81, member A (FAM81A), mRNA [NM_152450] | 10.056565 |
| FAM84A | Homo sapiens family with sequence similarity 84, member A (FAM84A), mRNA [NM_145175] | 5.9335413 |
| FCN1 | Homo sapiens ficolin (collagen/fibrinogen domain containing) 1 (FCN1), mRNA [NM_002003] | 5.719947 |
| FCRL1 | Homo sapiens Fc receptor-like 1 (FCRL1), transcript variant 1, mRNA [NM_052938] | 12.248644 |
| FGF10 | Homo sapiens fibroblast growth factor 10 (FGF10), mRNA [NM_004465] | 5.7203684 |
| FGL2 | Homo sapiens fibrinogen-like 2 (FGL2) mRNA [NM_006682] | 8.028772 |
| FILIP1 | Homo sapiens filamin A interacting protein 1 (FILIP1), mRNA [NM_015687] | 16.517225 |
| FILIP1 | Homo sapiens filamin A interacting protein 1 (FILIP1), mRNA [NM_015687] | 46.036945 |
| FLJ31485 | Homo sapiens uncharacterized LOC440119 (FLJ31485), non-coding RNA [NR_033834] | 7.824378 |
| FLJ38773 | Homo sapiens cDNA FLJ38773 fis, clone KIDNE2018071, [AK096092] | 5.1739235 |
| FOXQ1 | Homo sapiens forkhead box Q1 (FOXQ1), mRNA [NM_033260] | 7.165542 |
| FSD2 | Homo sapiens mRNA; cDNA DKFZp451H129 (from clone DKFZp451H129), [AL833295] | 12.632027 |
| GABRB1 | Homo sapiens gamma-aminobutyric acid (GABA) A receptor, beta 1 (GABRB1), mRNA [NM_000812] | 15.691926 |
| GABRE | Homo sapiens gamma-aminobutyric acid (GABA) A receptor, epsilon (GABRE), mRNA [NM_004961] | 5.660591 |
| GABRQ | gamma-aminobutyric acid (GABA) receptor, theta [Source: HGNC Symbol; Acc: 14454] | 7.757458 |
| | [ENST00000370306] | |
| GBP3 | Homo sapiens guanylate binding protein 3 (GBP3), mRNA [NM_018284] | 6.0336056 |
| GBP3 | Homo sapiens guanylate binding protein 3 (GBP3), mRNA [NM_018284] | 7.7593327 |
| GBP4 | Homo sapiens guanylate binding protein 4 (GBP4), mRNA [NM_052941] | 16.179169 |
| GBP5 | Homo sapiens guanylate binding protein 5 (GBP5), transcript variant 1, mRNA [NM_052942] | 21.121655 |
| GDAP1L1 | Homo sapiens ganglioside-induced differentiation-associated protein 1-like 1 (GDAP1L1), mRNA | 18.004025 |
| | [NM_1324034] | |
| GDPD1 | Homo sapiens glvcerophosphodiester phosphodiesterase domain containing 1 (GDPD1), transcript variant 1, | 8.417395 |
| | mRNA [NM_182569] | |
| GH2 | Homo sapiens growth hormone 2 (GH2), transcript variant 3, mRNA [NM_022558] | 6.60331 |
| GJB1 | Homo sapiens gap junction protein, beta 1, 32 kDa (GJB1), transcript variant 2, mRNA [NM_000166] | 7.0337234 |
| GLIPR1L2 | Homo sapiens GLI pathogenesis-related 1 like 2 (GLIPR1L2), mRNA [NM_152436] | 9.738023 |
| GLYATL1 | Homo sapiens glycine-N-acyltransferase-like 1 (GLYATL1) transcript variant 1 mRNA [NM_080661] | 8.92534 |
| GNAO1 | Homo sapiens cDNA clone IMAGE: 4181241, [BC012202] | 70.77483 |
| GPC6 | Homo sapiens glypican 6 (GPC6), mRNA [NM_005708] | 5.6233478 |
| GPNMB | Homo sapiens glycoprotein (transmembrane) nmb (GPNMB), transcript variant 1, mRNA [NM_001005340] | 28.65461 |
| GREM2 | Homo sapiens gremlin 2 (GREM2), mRNA [NM_022469] | 5.2581577 |
| GREM2 | Homo sapiens gremlin 2 (GREM2), mRNA [NM_022469] | 8.499975 |
| GRIK2 | Homo sapiens glutamate receptor, ionotropic, kainate 2 (GRIK2), transcript variant 1, mRNA [NM_021956] | 9.429901 |
| GRIN2A | Homo sapiens glutamate receptor, ionotropic N-methyl D-aspartate 2A (GRIN2A) transcript variant 2, | 6.404186 |
| | mRNA [NM_000833] | |
| HBD | Homo sapiens hemoglobin, delta (HBD), mRNA [NM_000519] | 7.0085983 |
| HCG23 | Homo sapiens HLA complex group 23 (HCG23), non-coding RNA [NR_044996] | 6.039699 |
| HDAC4 | Homo sapiens histone deacetvlase 4 (HDAC4), mRNA [NM_006037] | 5.20067 |
| HFE | Homo sapiens hemochromatosis (HFE), transcript variant 11, mRNA [NM_139011] | 5.802557 |
| HIST3H2BB | Homo sapiens cDNA FLJ33901 fis, clone CTONG2008321, highly similar to HISTONE H2B F, | 9.457097 |
| | [AK091220] | |
| HLA-DMB | major histocompatibility complex class II DM beta [Source: HGNC Symbol; Acc: 4935] | 8.631182 |
| | [ENST00000547478] | |
| HMCN1 | Homo sapiens hemicentin 1 (HMCN1), mRNA [NM_031935] | 6.0087633 |
| HOXB2 | Homo sapiens homeobox B2 (HOXB2) mRNA [NM_002145] | 7.223098 |
| HOXB2 | Homo sapiens homeobox B2 (HOXB2), mRNA [NM_002145] | 12.996367 |
| HSD17B2 | Homo sapiens hydroxysteroid (17-beta) dehydrogenase 2 (HSD17B2), mRNA [NM_002153] | 8.057347 |
| HTR4 | Homo sapiens 5-hydroxytryptamine (serotonin) receptor 4 (HTR4), transcript variant i, mRNA | 10.306236 |
| | [NM_001040173] | |
| IGFBP3 | Homo sapiens insulin-like growth factor binding protein 3 (IGFBP3), transcript variant 1, mRNA | 5.8157325 |
| | [NM_001013398] | |
| IGFBP3 | Homo sapiens insulin-like growth factor binding protein 3 (IGFBP3), transcript variant 1, mRNA | 7.741202 |
| | [NM_001013398] | |
| IGFBP5 | Homo sapiens insulin-like growth factor binding protein 5 (IGFBP5), mRNA [NM_000599] | 5.1364026 |
| IGFBP5 | Homo sapiens insulin-like growth factor binding protein 5 (IGFBP5), mRNA [NM_000599] | 5.4666533 |
| IGSF23 | Homo sapiens immunoglobulin superfamily, member 23 (IGSF23), mRNA [NM_001205280] | 9.525644 |
| IL18R1 | Homo sapiens interleukin 18 receptor 1 (IL18R1), mRNA [NM_003855] | 10.403711 |
| IL32 | Homo sapiens interleukin 32 (IL32) transcript variant 1 mRNA [NM_001012631] | 7.4202857 |
| IL7 | Homo sapiens interleukin 7 (IL7), transcript variant 1, mRNA [NM_000880] | 11.396346 |
| IRF5 | Homo sapiens interferon regulatory factor 5 (IRF5), transcript variant 3, mRNA [NM_001098627] | 11.967017 |
| ITGB8 | Homo sapiens integrin, beta 8 (ITGB8), mRNA [NM_002214] | 5.975307 |
| ITGB8 | Homo sapiens integrin, beta 8 (ITGB8), mRNA [NM_002214] | 6.0024295 |
| ITGB8 | Homo sapiens integrin, beta 8 (ITGB8), mRNA [NM_002214] | 12.839784 |
| JMJD5 | Homo sapiens cDNA FLJ61151 complete cds, [AK298410] | 7.646885 |
| KCNK12 | Homo sapiens potassium channel subfamily K, member 12 (KCNK12), mRNA [NM_022055] | 6.1507177 |
| KCTD4 | Homo sapiens potassium channel tetramerisation domain containing 4 (KCTD4), mRNA [NM_198404] | 6.600999 |
| KGFLP1 | Homo sapiens fibroblast growth factor 7 pseudogene (KGFLP1), non-coding RNA [NR_003674] | 5.0799804 |
| KIAA1656 | Homo sapiens mRNA for K1AA1656 protein, partial cds, [AB051443] | 10.032778 |
| KIAA1908 | Homo sapiens uncharacterized LOC114796 (KIAA1908), transcript variant 1, non-coding RNA [NR_027329] | 7.4855485 |
| KLF6 | Kruppel-like factor 6 [Source: HGNC Symbol: Acc: 2235] [ENST00000469435] | 16.583063 |
| KLHL20 | kelch-like 20 (Drosophila) [Source: HGNC Symbol; Acc: 25056] [ENST00000493170] | 5.261228 |
| KNDC1 | Homo sapiens kinase non-catalytic C-lobe domain (KIND) containing 1 (KNDC1), transcript variant 1, | 7.535085 |
| | mRNA [NM_152643] | |
| KPNA4 | Homo sapiens karyopherin alpha 4 (importin alpha 3) (KPNA4), mRNA [NM_002268] | 9.611041 |
| KRTAP1-3 | Homo sapiens keratin associated protein 1-3 (KRTAP1-3), mRNA [NM_030966] | 20.244638 |
| KRTAP13-1 | Homo sapiens keratin associated protein 13-1 (KRTAP13-1), mRNA [NM_181599] | 15.870147 |
| KYNU | Homo sapiens kynureninase (KYNU), transcript variant 1, mRNA [NM_003937] | 6.6586976 |
| KYNU | Homo sapiens kynureninase (KYNU), transcript variant 2, mRNA [NM_001032998] | 8.040255 |
| LAMA4 | Homo sapiens laminin, alpha 4 (LAMA4), transcript variant 2 mRNA [NM_002290] | 5.060427 |
| LENG9 | Homo sapiens leukocyte receptor cluster (LRC) member 9 (LENG9), mRNA [NM_198988] | 7.067042 |
| LINC00261 | Homo sapiens long intergenic non-protein coding RNA 261 (LINC00261) non-coding RNA [NR_001558] | 32.20773 |
| LIPG | Homo sapiens lipase, endothelial (LIPG), mRNA [NM_006033] | 189.19264 |
| LOC100130071 | PREDICTED: Homo sapiens GSQS6193 (LOC100130071), miscRNA [XR_109863] | 34.58081 |
| LOC100133130 | Homo sapiens clone FLB4246 PRO1102 mRNA, complete cds, [AF130105] | 6.199081 |
| LOC100505619 | Homo sapiens uncharacterized LOC100505619 (LOC100505619) non-coding RNA [NR_038233] | 14.224171 |
| LOC100506310 | chromosome 1 open reading frame 167 [Source: HGNC Symbol; Acc: 25262] [ENST00000433342] | 10.584649 |
| LOC100506310 | chromosome 1 open reading frame 167 [Source: HGNC Symbol; Acc: 25262] [ENST00000433342] | 11.197517 |
| LOC100507421 | Homo sapiens transmembrane protein 178-like (LOC100507421), mRNA [NM_001195278] | 50.283722 |
| LOC100652730 | PREDICTED: Homo sapiens hypothetical LOC100652730 (LOC100652730), miscRNA [XR_132670] | 21.34663 |
| LOC221442 | Homo sapiens adenylate cyclase 10 (soluble) pseudogene (LOC221442), non-coding RNA [NR_026938] | 6.6282353 |
| LOC284072 | Homo sapiens cDNA FLJ38084 fis, clone CTONG2016499, [AK095403] | 50.38662 |
| LOC286272 | Homo sapiens cDNA FLJ10077 fis, clone HEMBA1001864, [AK000939] | 5.052828 |
| LOC647946 | Homo sapiens uncharacterized LOC647946 (LOC647946), non-coding RNA [NR_024391] | 34.53928 |
| LOC84931 | Homo sapiens uncharacterized LOC84931 (LOC84931), non-coding RNA [NR_027181] | 5.500767 |
| LPAR3 | Homo sapiens lysophosphatidic acid receptor 3 (LPAR3), mRNA [NM_012152] | 5.105164 |
| LPAR3 | Homo sapiens lysophosphatidic acid receptor 3 (LPAR3), mRNA [NM_012152] | 6.1999454 |
| LPHN3 | Homo sapiens latrophilin 3 (LPHN3), mRNA [NM_015236] | 12.726456 |
| LPIN1 | lipin 1 [Source: HGNC Symbol; Acc: 13345] [ENST00000460096] | 15.806569 |
| LRP1B | Homo sapiens low density lipoprotein receptor-related protein 1B (LRP1B), mRNA [NM_018557] | 12.464852 |
| LRRC19 | Homo sapiens leucine rich repeat containing 19 (LRRC19), mRNA [NM_022901] | 96.5277 |
| LRRC3 | Homo sapiens leucine rich repeat containing 3 (LRRC3), mRNA [NM_030891] | 5.0677733 |
| LY75 | Homo sapiens lymphocyte antigen 75 (LY75), mRNA [NM_002349] | 9.694963 |
| LYVE1 | Homo sapiens lymphatic vessel endothelial hyaluronan receptor 1 (LYVE1), mRNA [NM_006691] | 7.6508794 |
| MAB21L1 | Homo sapiens mab-21-like 1 (C. elegans) (MAB21L1), mRNA [NM_005584] | 5.0701036 |
| MECOM | Homo sapiens MDS1 and EVI1 complex locus (MECOM), transcript variant 2, mRNA [NM_005241] | 5.1384163 |
| MEIS2 | Homo sapiens Meis homeobox 2 (MEIS2), transcript variant d, mRNA [NM_170676] | 9.917238 |
| MGP | Homo sapiens matrix Gla protein (MGP), transcript variant 2, mRNA [NM_000900] | 27.222235 |
| MIAT | Homo sapiens myocardial infarction associated transcript (non-protein coding) (MIAT), transcript variant 1, | 5.6434402 |
| | non-coding RNA [NR_003491] | |
| MIAT | Homo sapiens myocardial infarction associated transcript (non-protein coding) (MIAT), transcript variant 1, | 6.6361594 |
| | non-coding RNA [NR_003491] | |
| MIR1245A | Homo sapiens microRNA 1245 (MIR1245), microRNA [NR_031647] | 7.491962 |
| MMP10 | Homo sapiens matrix metallopeptidase 10 (stromelysin 2) (MMP10), mRNA [NM_002425] | 18.814783 |
| MX2 | Homo sapiens myxovirus (influenza virus) resistance 2 (mouse) (MX2), mRNA [NM_002463] | 9.206769 |
| MYCL1 | Homo sapiens v-myc myelocytomatosis viral oncogene homolog 1, lung carcinoma derived (avian) | 5.310897 |
| | (MYCL1), transcript variant 3, mRNA [NM_005376] | |
| MYO1G | Homo sapiens myosin IG (MYO1G), mRNA [NM_033054] | 49.64526 |
| MYO1H | Homo sapiens cDNA FLJ37587 fis, clone BRCOC2005951, moderately similar to B. taurus myosin IB | 36.559258 |
| | mRNA, [AK094906] | |
| MYT1L | Homo sapiens myelin transcription factor 1-like (MYT1L), mRNA [NM_015025] | 22.714705 |
| NETO1 | Homo sapiens neuropilin (NRP) and tolloid (TLL)-like 1 (NETO1), transcript variant 3, mRNA | 9.145789 |
| | [NM_138966] | |
| NFIB | Homo sapiens nuclear factor I/B (NFIB), transcript variant 3, mRNA [NM_005596] | 5.8848667 |
| NKX6-3 | Homo sapiens cDNA FLJ25169 fis, clone CBR08739, [AK057898] | 15.744906 |
| NRSN1 | Homo sapiens neurensin 1 (NRSN1), mRNA [NM_080723] | 7.9309936 |
| NTRK1 | Homo sapiens neurotrophic tyrosine kinase, receptor, type 1 (NTRK1), transcript variant 2, mRNA | 8.465271 |
| | [NM_002529] | |
| NTRK1 | Homo sapiens neurotrophic tyrosine kinase, receptor, type 1 (NTRK1), transcript variant 2, mRNA | 9.777625 |
| | [NM_002529] | |
| ODAM | Homo sapiens odontogenic, ameloblast asssociated (ODAM), mRNA [NM_017855] | 5.278667 |
| OGN | Homo sapiens osteoglycin (OGN), transcript variant 1, mRNA [NM_033014] | 6.186644 |
| OR12D3 | Homo sapiens olfactory receptor, family 12, subfamily D, member 3 (OR12D3), mRNA [NM_030959] | 5.8602066 |
| OR2T5 | Homo sapiens olfactory receptor family 2 subfamily T, member 5 (OR2T5) mRNA [NM_001004697] | 7.274157 |
| OR8J1 | Homo sapiens olfactory receptor family 8 subfamily J, member 1 (OR8J1) mRNA [NM_001005205] | 5.675051 |
| OXTR | Homo sapiens oxytocin receptor (OXTR), mRNA [NM_000916] | 5.4097543 |
| PALM2 | Homo sapiens paralemmin 2 (PALM2), transcript variant 1, mRNA [NM_053016] | 6.8525114 |
| PAPPA | Homo sapiens pregnancy-associated plasma protein A, pappalysin 1 (PAPPA), mRNA [NM_002581] | 8.157425 |
| PCDH20 | Homo sapiens protocadherin 20 (PCDH20), mRNA [NM_022843] | 6.5457754 |
| PCDHGAS | Homo sapiens protocadherin gamma subfamily A, 5 (PCDHGA5), transcript variant 2, mRNA [NM_032054] | 11.57362 |
| PDE11A | Homo sapiens phosphodiesterase 11A (PDE11A), transcript variant 4, mRNA [NM_016953] | 10.867444 |
| PDE11A | Homo sapiens phosphodiesterase 11A (PDE11A), transcript variant 4, mRNA [NM_016953] | 14.052832 |
| PDLIM3 | Homo sapiens PDZ and LIM domain 3 (PDLIM3), transcript variant 1, mRNA [NM_014476] | 7.2054434 |
| PDZRN3 | Homo sapiens PDZ domain containing ring finger 3, mRNA (cDNA clone IMAGE: 4639477), complete cds, | 5.5011473 |
| | [BC014432] | |
| PDZRN4 | Homo sapiens PDZ domain containing ring finger 4 (PDZRN4), transcript variant 2, mRNA [NM_013377] | 8.555303 |
| PENK | Homo sapiens proenkephalin (PENK), transcript variant 2, mRNA [NM_006211] | 7.6583657 |
| PF4 | Homo sapiens platelet factor 4 (PF4), mRNA [NM_002619] | 10.509349 |
| PF4V1 | Homo sapiens platelet factor 4 variant 1 (PF4V1), mRNA [NM_002620] | 33.466225 |
| PHF20 | Homo sapiens PHD finger protein 20 (PHF20) mRNA [NM_016436] | 9.694313 |
| PIAS4 | Homo sapiens protein inhibitor of activated STAT, 4 (PEAS4), mRNA [NM_015897] | 22.315287 |
| PITPNC1 | Homo sapiens phosphatidylinositol transfer protein, cytoplasmic 1 (PITPNC1), transcript variant 2, mRNA | 7.40878 |
| | [NM_181671] | |
| PLAC4 | Homo sapiens placenta-specific 4 (PLAC4), mRNA [NM_182832] | 6.6760406 |
| PLK5 | Homo sapiens polo-like kinase 5 (PLK5), mRNA [NM_001243079] | 12.394606 |
| PODN | Homo sapiens podocan (PODN) transcript variant 1 mRNA [NM_153703] | 5.9192147 |
| POFUT2 | Homo sapiens protein O-fucosyltransferase 2 (POFUT2), transcript variant 3, mRNA [NM_133635] | 6.6909666 |
| POSTN | Homo sapiens periostin, osteoblast specific factor (POSTN), transcript variant 1, mRNA [NM_006475] | 13 452635 |
| PPHLN1 | Homo sapiens periphilin 1 (PPHLN1), transcript variant 5, mRNA [NM_201438] | 7.2769685 |
| PPL | Homo sapiens periplakin (PPL), mRNA [NM_002705] | 5.111492 |
| PRDM1 | Homo sapiens PR domain containing 1, with ZNF domain (PRDM1), transcript variant 1, mRNA | 11.060661 |
| | [NM_001198] | |
| PRSS35 | Homo sapiens protease serine 35 (PRSS35) transcript variant 2, mRNA [NM_153362] | 17.777374 |
| PRUNE2 | Homo sapiens prune homolog 2 (Drosophila) (PRUNE2), mRNA [NM_015225] | 5.215551 |
| PTGS1 | Homo sapiens prostaglandin-endoperoxide synthase 1 (prostaglandin G/H synthase and cyclooxygenase) | 5.351385 |
| | (PTGS1), transcript variant 1, mRNA [NM_000962] | |
| PTPRE | Homo sapiens protein tyrosine phosphatase receptor type, E (PTPRE) transcript variant 1 mRNA | 5.592387 |
| | [NM_006504] | |
| RAB7B | Homo sapiens RAB7B, member RAS oncogene family (RAB7B), transcript variant 1, mRNA [NM_177403] | 7.147107 |
| RARRES2 | Homo sapiens retinoic acid receptor responder (tazarotene induced) 2 (RARRES2), mRNA [NM_002889] | 9.080296 |
| RASL11A | Homo sapiens RAS-like, family 11, member A (RASL11A), mRNA [NM_206827] | 5.9116917 |
| RASL11B | Homo sapiens RAS-like family 11, member B (RASL11B), mRNA [NM_023940] | 6.3131185 |
| RAVER2 | ribonucleoprotein, PTB-binding 2 [Source: HGNC Symbol; Acc: 25577] [ENST00000418058] | 10.11057 |
| RBMY2FP | Homo sapiens RNA binding motif protein, Y-linked, family 2, member F pseudogene (RBMY2FP), | 47.26753 |
| | non-coding RNA [NR_002193] | |
| RDH10 | Homo sapiens retinal dehydrogenase 10 (all-trans) (RDH10), mRNA [NM_172037] | 8.269352 |
| RHOV | Homo sapiens ras homolog gene family member V (RHOV), mRNA [NM_133639] | 14.287875 |
| RIMKLA | Homo sapiens ribosomal modification protein rimK-like family member A (RIMKLA) mRNA [NM_173642] | 5.5233345 |
| ROPN1 | Homo sapiens rhophilin associated tail protein 1 (ROPN1), mRNA [NM_017578] | 7.195612 |
| RSPO2 | Homo sapiens R-spondin 2 (RSPO2) mRNA [NM_178565] | 7.1980314 |
| SAMD3 | Homo sapiens sterile alpha motif domain containing 3 (SAMD3), transcript variant 1, mRNA | 5.4209805 |
| | [NM_001017373] | |
| SCG2 | Homo sapiens secretogranin II (SCG2) mRNA [NM_003469] | 6.993405 |
| SDPR | Homo sapiens serum deprivation response (SDPR), mRNA [NM_004657] | 5.0143037 |
| SEMA3G | Homo sapiens sema domain, immunoglobulin domain (Ig), short basic domain, secreted, (semaphorin) 3G | 25.342768 |
| | (SEMA3G), mRNA [NM_020163] | |
| SEMG2 | Homo sapiens semenogelin II (SEMG2), mRNA [NM_003008] | 83.793274 |
| SERINC4 | serine incorporator 4 [Source: HGNC Symbol; Acc: 32237] [ENST00000319327] | 13.489073 |
| SFRP2 | Homo sapiens secreted frizzled-related protein 2 (SFRP2), mRNA [NM_003013] | 10.7199335 |
| SFRP2 | Homo sapiens secreted frizzled-related protein 2 (SFRP2), mRNA [NM_003013] | 12.669123 |
| SFRP2 | Homo sapiens secreted frizzled-related protein 2 (SFRP2), mRNA [NM_003013] | 14.794545 |
| SFTPA1 | Homo sapiens surfactant protein A1 (SFTPA1), transcript variant 1, mRNA [NM_005411] | 5.4909563 |
| SHANK1 | Homo sapiens SH3 and multiple ankyrin repeat domains 1 (SHANK1), mRNA [NM_016148] | 5.1189075 |
| SHANK2 | Homo sapiens SH3 and multiple ankyrin repeat domains 2 (SHANK2), transcript variant 1, mRNA | 6.0359373 |
| | [NM_012309] | |
| SKI | Homo sapiens v-ski sarcoma viral oncogene homolog (avian) (SKI), mRNA [NM_003036] | 6.3805013 |
| SKINTL | Homo sapiens Skint-like, pseudogene (SKINTL), non-coding RNA [NR_026749] | 7.7992816 |
| SLA | Homo sapiens Src-like-adaptor (SLA), transcript variant 1, mRNA [NM_001045556] | 7.620312 |
| SLC14A1 | Homo sapiens solute carrier family 14 (urea transporter), member 1 (Kidd blood group) (SLC14A1), | 5.177023 |
| | transcript variant 4, mRNA [NM_001146037] | |
| SLC15A1 | Homo sapiens solute carrier family 15 (oligopeptide transporter), member 1 (SLC15A1), mRNA | 57.607304 |
| | [NM_005073] | |
| SLC16A6 | Homo sapiens solute carrier family 16, member 6 (monocarboxylic acid transporter 7) (SLC16A6), transcript | 7.3237977 |
| | variant 2, mRNA [NM_004694] | |
| SLC16A6 | Homo sapiens solute carrier family 16, member 6 (monocarboxylic acid transporter 7) (SLC16A6), transcript | 8.627408 |
| | variant 2, mRNA [NM_004694] | |
| SLC22A31 | Homo sapiens solute carrier family 22, member 31 (SLC22A31), mRNA [NM_001242757] | 13.826827 |
| SLC26A1 | Homo sapiens solute carrier family 26 (sulfate transporter), member 1 (SLC26A1), transcript variant 2, | 15.7760315 |
| | mRNA [NM_134425] | |
| SLC6A1 | Homo sapiens solute carrier family 6 (neurotransmitter transporter, GABA), member 1 (SLC6A1), mRNA | 6.079788 |
| | [NM_003042] | |
| SLC7A14 | Homo sapiens solute carrier family 7 (orphan transporter), member 14 (SLC7A14), mRNA [NM_020949] | 5.0602446 |
| SLC7A14 | Homo sapiens solute carrier family 7 (orphan transporter), member 14 (SLC7A14), mRNA [NM_020949] | 14.438024 |
| SLC9A9 | solute carrier amily 9 (sodium/hydrogen exchanger), member 9 [Source: HGNC Symbol; Acc: 20653] | 10.170171 |
| | [ENST00000498717] | |
| SNCA | Homo sapiens synuclein alpha (non A4 component of amyloid precursor) (SNCA), transcript variant 1, | 9.417178 |
| | mRNA [NM_000345] | |
| SNED1 | Homo sapiens sushi, nidogen and EGF-like domains 1 (SNED1), mRNA [NM_001080437] | 6.55686 |
| SNED1 | Homo sapiens sushi, nidogen and EGF-like domains 1 (SNED1), mRNA [NM_001080437] | 11.57066 |
| SNX10 | Homo sapiens sorting nexin 10 (SNX10), transcript variant 2, mRNA [NM_013322] | 6.5760717 |
| SNX10 | Homo sapiens sorting nexin 10 (SNX10), transcript variant 2 mRNA [NM_013322] | 8.102584 |
| SOX2 | Homo sapiens SRY (sex determining region Y)-box 2 (SOX2), mRNA [NM_003106] | 5.588049 |
| SPATA16 | Homo sapiens spermatogenesis associated 16 (SPATA16) mRNA [NM_031955] | 6.114361 |
| SPDYE3 | Homo sapiens speedy homolog E3 (Xenopus laevis) (SPDYE3), mRNA [NM_001004351] | 6.2118874 |
| SPON2 | Homo sapiens spondin 2, extracellular matrix protein (SPON2), transcript variant 1, mRNA [NM_012445] | 21.017044 |
| SPP2 | Homo sapiens secreted phosphoprotein 2, 24 kDa (SPP2), mRNA [NM_006944] | 6.7812304 |
| SPRR2D | Homo sapiens small proline-rich protein 2D (SPRR2D), mRNA [NM_006945] | 6.5720673 |
| SPRR4 | Homo sapiens small proline-rich protein 4 (SPRR4) mRNA [NM_173080] | 30.993826 |
| SPRY1 | Homo sapiens sprouty homolog 1, antagonist of FGF signaling (Drosophila) (SPRY1), transcript variant 2, | 7.310849 |
| | mRNA [NM_199327] | |
| SST | Homo sapiens somatostatin (SST), mRNA [NM_001048] | 6.230418 |
| ST8SIA1 | Homo sapiens ST8 alpha-N-acetyl-neuraminide alpha-2,8-sialyltransferase 1 (ST8SIA1), mRNA | 7.1481137 |
| | [NM_003034] | |
| SYCE1 | Homo sapiens synaptonemal complex central element protein 1 (SYCE1) transcript variant 4 mRNA | 8.670044 |
| | [NM_001143764] | |
| TAAR9 | Homo sapiens trace amine associated receptor 9 (gene/pseudogene) (TAAR9), mRNA [NM_175057] | 24.728695 |
| TACR1 | Homo sapiens tachykinin receptor 1 (TACR1), transcript variant short, mRNA [NM_015727] | 8.226762 |
| TANC2 | Homo sapiens cDNA FLJ10215 fis, clone HEMBA1006737, [AK001077] | 6.404711 |
| TFPI2 | tissue factor pathway inhibitor 2 [Source: HGNC Symbol; Acc: 11761] [ENST00000222543] | 7.8648047 |
| TFPI2 | Homo sapiens tissue factor pathway inhibitor 2 (TFPI2), mRNA [NM_006528] | 11.574308 |
| TGFB3 | Homo sapiens transforming growth factor, beta 3 (TGFB3), mRNA [NM_003239] | 5.0866065 |
| THPO | Homo sapiens thrombopoietin (THPO), transcript variant 1, mRNA [NM_000460] | 12.783364 |
| THSD7A | Homo sapiens thrombospondin, type I, domain containing 7A (THSD7A), mRNA [NM_015204] | 5.149231 |
| TM4SF1 | Homo sapiens transmembrane 4 L six family member 1 (TM4SF1), mRNA [NM_014220] | 12.421069 |
| TMEM100 | Homo sapiens transmembrane protein 100 (TMEM100), transcript variant 2, mRNA [NM_018286] | 7.55566 |
| TMEM176A | Homo sapiens transmembrane protein 176A (TMEM176A), mRNA [NM_018487] | 14.376767 |
| TMEM176B | Homo sapiens transmembrane protein 176B (TMEM176B), transcript variant 1, mRNA [NM_014020] | 15.972688 |
| TMEM223 | transmembrane protein 223 [Source: HGNC Symbol; Acc: 28464] [ENST00000527073] | 12.1265135 |
| TNFAIP6 | Homo sapiens tumor necrosis factor, alpha-induced protein 6 (TNFAIP6), mRNA [NM_007115] | 5.260216 |
| TNFRSF10C | Homo sapiens tumor necrosis factor receptor superfamily, member 10c, decoy without an intracellular | 5.2102785 |
| | domain (TNFRSF10C), mRNA [NM_003841] | |
| TNFRSF8 | Homo sapiens tumor necrosis factor receptor superfamily, member 8 (TNFRSF8), transcript variant 1, | 45.364815 |
| | mRNA [NM_001243] | |
| TPD52L3 | Homo sapiens tumor protein D52-like 3 (TPD52L3), transcript variant 1, mRNA [NM_033516] | 9.196981 |
| TPH2 | Homo sapiens tryptophan hydroxylase 2 (TPH2), mRNA [NM_173353] | 14.183445 |
| TPTE | Homo sapiens transmembrane phosphatase with tensin homology (TPTE), transcript variant 3, mRNA | 5.4394703 |
| | [NM_199260] | |
| TRIL | Homo sapiens TLR4 interactor with leucine-rich repeats (TRIL), mRNA [NM_014817] | 5.8490143 |
| TRPA1 | Homo sapiens transient receptor potential cation channel, subfamily A, member 1 (TRPA1), mRNA | 12.133277 |
| | [NM_007332] | |
| TRPA1 | Homo sapiens transient receptor potential cation channel, subfamily A, member 1 (TRPA1), mRNA | 15.086606 |
| | [NM_007332] | |
| TSPAN18 | Homo sapiens tetraspanin 18 (TSPAN18), mRNA [NM_130783] | 6.5376697 |
| UGT2B7 | Homo sapiens UDP glucuronosyltransferase 2 family, polypeptide B7 (UGT2B7), mRNA [NM_001074] | 9.120112 |
| UGT3A1 | Homo sapiens UDP glycosyltransferase 3 family, polypeptide A1 (UGT3A1), transcript variant 1, mRNA | 5.5417686 |
| | [NM_152404] | |
| UPK3A | Homo sapiens uroplakin 3A (UPK3A) transcript variant 1, mRNA [NM_006953] | 7.208652 |
| VSTM4 | Homo sapiens V-set and transmembrane domain containing 4 (VSTM4) transcript variant 1, mRNA | 5.7906747 |
| | [NM_001031746] | |
| WDR64 | Homo sapiens WD repeat domain 64 (WDR64) mRNA [NM_144625] | 5.002266 |
| WHSC1 | Homo sapiens Wolf-Hirschhom syndrome candidate 1 (WHSC1), transcript variant 7, mRNA [NM_133334] | 5.3172607 |
| WIF1 | Homo sapiens WNT inhibitory factor 1 (WIF1), mRNA [NM_007191] | 18.270514 |
| WNT16 | Homo sapiens wingless-type MMTV integration site family, member 16 (WNT16), transcript variant 1, | 10.549207 |
| | mRNA [NM_057168] | |
| WNT9A | wingless-type MMTV integration site family, member 9A [Source: HGNC Symbol; Acc: 12778] | 6.0163407 |
| | [ENST00000272164] | |
| XG | Homo sapiens Xe blood group (XG), transcript variant 1, mRNA [NM_175569] | 6.842992 |
| YPEL4 | Homo sapiens yippee-like 4 (Drosophila) (YPEL4), mRNA [NM_145008] | 5.38302 |
| ZDHHC22 | zinc finger, DHHC-type containing 22 [Source: HGNC Symbol; Acc: 20106] [ENST00000555327] | 59.691048 |
| ZNF175 | Homo sapiens zinc finger protein 175, mRNA (cDNA clone IMAGE: 4301632), partial cds, [BC007778] | 11.4099 |
| ZNF254 | Homo sapiens zinc finger protein 254 (ZNF254), mRNA [NM_203282] | 6.746976 |
| ZNF385B | Homo sapiens zinc finger protein 385B (ZNF385B), transcript variant 1, mRNA [NM_152520] | 5.410112 |
| ZNF385D | Homo sapiens zinc finger protein 385D (ZNF385D), mRNA [NM_024697] | 8.568527 |
| ZNF385D | zinc finger protein 385D [Source: HGNC Symbol; Acc: 26191] [ENST00000281523] | 28.338871 |
| ZNF618 | zinc finger protein 618 [Source: HGNC Symbol; Acc: 29416] [ENST00000374126] | 5.245748 |
| ZNF708 | Homo sapiens zinc finger protein 708 (ZNF708) mRNA [NM_021269] | 53.38155 |
| ZP3 | Homo sapiens zona pellucida glycoprotein 3 (sperm receptor) (ZP3), transcript variant 2, mRNA | 6.9537215 |
| | [NM_007155] | |

The dental pulp stem cell to be used in producing a graft material for treating nerve damage according to the specification may be a dental pulp stem cell in which the expression levels of one type or more, two types or more, 3 types or more, 4 types or more, 5 types or more, 6 types or more, 7 types or more, 8 types or more, 9 types or more, 10 types or more, 11 types or more, 12 types or more, 13 types or more, 14 types or more, 15 types or more, 16 types or more, 17 types or more, 18 types or more, 19 types or more, 20 types or more, or 21 types of genes selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg, and Cbln2 are each 5 times or more, 10 times or more, 20 times or more, 30 times or more, 40 times or more, 50 times or more, 60 times or more, 70 times or more, 80 times or more, 90 times or more, 95 times or more, or 100 times or more, each are as high as an average expression level thereof in the dental pulp stem cells.

Among two or more groups of dental pulp stem cells, a group of dental pulp stem cells in which the gene expression level of the group of genes listed in Table 1 is high may be selected and used as the dental pulp stem cell to be used in producing a graft material for treating nerve damage according to the specification.

More specifically, a group of dental pulp stem cells in which the expression levels of the genes of 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 98% or more of the group of genes described in Table 1, each are 5 times or more, 10 times or more, 20 times or more, 30 times or more, 40 times or more, 50 times or more, 60 times or more, 70 times or more, 80 times or more, 90 times or more, 95 times or more, or 100 times or more as high as other groups of cells, may be selected and used.

Among two types or more groups of dental pulp stem cells, a group of dental pulp stem cells in which the expression levels of one type or more, two types or more, 3 types or more, 4 types or more, 5 types or more, 6 types or more, 7 types or more, 8 types or more, 9 types or more, 10 types or more, 11 types or more, 12 types or more, 13 types or more, 14 types or more, 15 types or more, 16 types or more, 17 types or more, 18 types or more, 19 types or more, 20 types or more, or 21 types of genes selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg, and Cbln2, each are 5 times or more, 10 times or more, 20 times or more, 30 times or more, 40 times or more, 50 times or more, 60 times or more, 70 times or more, 80 times or more, 90 times or more, 95 times or more, or 100 times or more as high as those of other groups of cells, may be selected and used as the dental pulp stem cell to be used in producing a graft material for treating nerve damage according to the specification.

In the specification, the “two types or more groups of dental pulp stem cells” refer to, for example, groups of dental pulp stem cells derived from two or more individuals; groups of the cells collected from a single individual at the intervals of a predetermined time; and groups of the cells derived from different teeth of a single individual. A group of cells may be a cell line proliferated from a single cell or a group of cells obtained by culturing a plurality of cells derived from a single individual.

In the specification, the type and level of gene expression can be examined by techniques known to those skilled in the art including Northern blotting, in-situ hybridization, RNAse protection assay and reverse transcription polymerase chain reaction (RT-PCR); however the techniques are not limited to these.

The dental pulp stem cell to be used in producing a graft material for treating nerve damage according to the specification may be a dental pulp stem cell in which the gene expression level of the group of genes listed in Table 2 is low compared to an average expression level in dental pulp stem cells.

In the specification, the “dental pulp stem cell, in which the gene expression level of the group of genes listed in Table 2 is low compared to an average expression level in dental pulp stem cells” refers to a dental pulp stem cell satisfying the following condition: when gene expression pattern was checked with respect to expression of the group of genes described in Table 2, the expression levels of genes corresponding to 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 98% or more of the group of genes described in Table 2, each are 5 times or more, 10 times or more, 20 times or more, 30 times or more, 40 times or more, 50 times or more, 60 times or more, 70 times or more, 80 times or more, 90 times or more, 95 times or more, or 100 times or more as low as the average expression level in dental pulp stem cells.

TABLE 2

| | | Times |
|---|---|---|
| GeneSymbol | Explanation [Genbank Accession Number] | (DP264/DP31) |
| ASHD13 | Homo sapiens abhydrolase domain containing 13 (ABHD13) mRNA [NM_032859] | 6.7370195 |
| ADRA2A | Homo sapiens adrenergic, alpha-2A-, receptor (ADRA2A), mRNA [NM_000681] | 5.5700407 |
| AK8 | Homo sapiens cDNA FLJ36014 fis, clone TESTI2016101, [AK093333] | 6.629828 |
| ANKRD32 | Homo sapiens ankyrin repeat domain 32 (ANKRD32), mRNA [NM_032290] | 9.894372 |
| AP1S3 | adaptor-related protein complex 1, sigma 3 subunit [Source: HGNC Symbol; Acc: 18971] | 24.408966 |
| | [ENST00000423110] | |
| ATP1B4 | Homo sapiens ATPase, Na+/K+ transporting beta 4 polypeptide (ATP164), transcript variant 2, mRNA | 26.121454 |
| | [NM_012069] | |
| ATP2A3 | Homo sapiens ATPase, Ca++ transporting, ubiquitous (ATP2A3), transcript variant 5, mRNA [NM_174953] | 5.542635 |
| AUTS2 | Homo sapiens autism susceptibility candidate 2 (AUTS2), transcript variant 3, mRNA [NM_001127232] | 5.9751472 |
| BET3L | Homo sapiens BET3 like (S. cerevisiae) (BET3L), mRNA [NM_001139444] | 11.572534 |
| BEX5 | Homo sapiens brain expressed, X-linked 5 (BEX5), transcript variant 1, mRNA [NM_001012978] | 10.4047575 |
| BHMT2 | Homo sapiens betaine--homocysteine S-methyltransferase 2 (BHMT2), transcript variant 1, mRNA | 27.803728 |
| | [NM_017614] | |
| BMP7 | Homo sapiens bone morphogenetic protein 7 (BMP7), mRNA [NM_001719] | 7.0865164 |
| C11orf85 | chromosome 11 open reading frame 85 [Source: HGNC Symbol; Acc: 27441] [ENST00000530735] | 7.796952 |
| C13orf30 | Homo sapiens chromosome 13 open reading frame 30 (C13orf30), mRNA [NM_182608] | 12.322237 |
| C19orf21 | Homo sapiens chromosome 19 open reading frame 21 (C19orf21), mRNA [NM_173481] | 10.701988 |
| C29orf71 | Homo sapiens chromosome 19 open reading frame 71 (C19orf71), mRNA [NM_001135580] | 12.649362 |
| C1orf168 | Homo sapiens chromosome 1 open reading frame 168 (C1orf168), rnRNA [NM)2101004303] | 7.22391 |
| C1orf88 | Homo sapiens chromosome 1 open reading frame 88 (C1orf138), mRNA [NM_181643] | 5.445106 |
| C22orf45 | Homo sapiens chromosome 22 open reading frame 45 (C22orf45), transcript variant 1, non-coding RNA | 6.600951 |
| | [NR_028484] | |
| C5orf52 | Homo sapiens chromosome 5 open reading frame 52 (C5orF52), mRNA [NM_001145132] | 6.120212 |
| C6orf10 | Homo sapiens chromosome 6 open reading frame 10 (C6orF10), mRNA [NM_006781] | 5.5895653 |
| CAMKMT | Homo sapiens chromosome 2 open reading frame 34, mRNA (cDNA clone IMAGE: 4673016), complete | 7.356156 |
| | cds, [BC029359] | |
| CAPN14 | Homo sapiens calpain 14 (CAPN14), mRNA [NM_001145122] | 9.117912 |
| CAPN6 | Homo sapiens calpain 6 (CAPN6), mRNA [NM_014289] | 7.772801 |
| CCDC27 | Homo sapiens coiled-coil domain containing 27 (CCDC27) mRNA [NM_152492] | 9.49639 |
| CCL3 | Homo sapiens mRNA for pLD7B peptide, complete cds, [D00044] | 13.145225 |
| CD80 | Homo sapiens CD80 molecule (CD80), mRNA [NM_005191] | 9.120709 |
| CELF2 | Homo sapiens CUGBP Elav-like family member 2 (CELF2) transcript variant 3 mRNA [NM_001025077] | 10.914636 |
| CLIC6 | Homo sapiens chloride intracellular channel 6 (CLIC6), nuclear gene encoding mitochondrial protein, mRNA | 8.431088 |
| | [NM_053277] | |
| CLVS1 | Homo sapiens clavesin 1 (CLVS1), mRNA [NM_173519] | 14.955474 |
| CPVL | Homo sapiens carboxypeptidase, vitellogenic-like (CPVL), transcript variant 2, mRNA [NM_01 9029] | 7.7193747 |
| CRYGD | Homo sapiens crystallin, gamma D, mRNA (cDNA clone MGC: 150917 IMAGE: 40125889), complete cds, | 18.035872 |
| | [BC117338] | |
| DBF4 | Homo sapiens DBF4 homolog (S. cerevisiae) (DBF4) mRNA [NM_006716] | 10.051733 |
| DFFA4 | Homo sapiens defensin alpha 4 corticostatin (DFFA4) mRNA [NM_001995] | 8.129146 |
| DPP10 | Homo sapiens dipeptidyl-peptidase 10 (non-functional) (DPP10), transcript variant 1, mRNA [NM_020868] | 5.980383 |
| DSG2 | Homo sapiens desmoglein 2 (DSG2), mRNA [NM_001943] | 8.120088 |
| DUOX1 | Homo sapiens mRNA; cDNA DKFZp434L0610 (from clone DKFZp434L0610); partial cds, [AL137592] | 5.4348574 |
| DUOX1 | Homo sapiens dual oxidase 1 (DUOX1), transcript variant 1, mRNA [NM_017434] | 6.6433687 |
| DYSF | Homo sapiens dysferlin, limb girdle muscular dystrophy 2B (autosomal recessive) (DYDF), transcript variant | 6.3100667 |
| | 8, mRNA [NM_003494] | |
| ESRRB | Human mRNA for steroid hormone receptor hERR2, [X51417] | 8.866751 |
| FAM13A | Homo sapiens mRNA; cDNA DKFZp686013152 (from clone DKFZp686013152), [RX647410] | 67.590775 |
| FAM27L | Homo sapiens family with sequence similarity 27-like (FAM27L), non-coding RNA [NR_028336] | 11.391356 |
| FAM9A | Homo sapiens family with sequence similarity 9, member A (FAM9A), transcript variant 2, mRNA | 5.6189966 |
| | [NM_174951] | |
| FLJ35024 | Homo sapiens uncharacterized LOC401491 (FLJ35024), non-coding RNA [NR_015375] | 5.126996 |
| FLJ38668 | Homo sapiens cDNA FLJ38668 fis, clone HLUNG2008439, [AK095987] | 18.998589 |
| FOXP2 | Homo sapiens forkhead box P2 (FOXP2), transcript variant 4, mRNA [NM_148900] | 15.616727 |
| FXR1 | Homo sapiens fragile X mental retardation, autosomal homolog 1 (FXR1), transcript variant 3, mRNA | 6.8956017 |
| | [NM_001013439] | |
| GABRA5 | Homo sapiens gamma-aminobutyric acid (GABA) A receptor alpha 5 (GABRA5), transcript variant 1, | 5.0026546 |
| | mRNA [NM_000810] | |
| GAFA3 | Homo sapiens FGF-2 activity-associated protein 3 (GAFA3) mRNA, complete cd [AF220235] | 58.031475 |
| GALNT3 | Homo sapiens UDP-N-acetyl-alpha-D-galactosamine: polypepride N-acetylgalactosaminyltransferase 3 | 10.374323 |
| | (GalNAc-T3) (GALNT3), mRNA [NM_004482] | |
| GEN1 | Homo sapiens Gen homolog 1, endonuclease (Drosophila), mRNA (cDNA clone IMAGE: 4513298) with | 5.0716524 |
| | apparent retained intron, [BC035863] | |
| GKN1 | Homo sapiens gastrokine 1 (GKN1), mRNA [NM_019617] | 76.34709 |
| GLT1D1 | Homo sapiens glycasyltransferase 1 domain containing 1 (GLT1D1), mRNA [NM_144669] | 8.2649555 |
| GNAL | Homo sapiens guanine nucleotide binding protein (G protein), alpha activating activity polypeptide, olfactory | 9.994541 |
| | type (GNAL), transcript variant 2, mRNA [NM_002071] | |
| GPR112 | Homo sapiens G protein-coupled receptor 112 (GPR112), mRNA [NM_153834] | 94.74006 |
| GPR27 | Homo sapiens G protein-coupled receptor 27 (GPR27), mRNA [NM_018971] | 179.00607 |
| GRM8 | Homo sapiens glutamate receptor, metabotropic 8 (GRM8), transcript variant 1, mRNA [NM_000845] | 7.08929 |
| GSG1L | Homo sapiens GSG1-like (GSG1L), transcript variant 2, mRNA [NM_144675] | 7.037393 |
| GSTT1 | Homo sapiens glutathione S-transferase theta 1 (GSTT1), mRNA [NM_000853] | 45.92394 |
| GSTT1 | Homo sapiens glutathione S-transferase theta 1 (GSTT1), mRNA [NM_000853] | 177.7299 |
| GUCY1A3 | Homo sapiens guanylate cyclase 1, soluble, alpha 3 (GUCY1A3), transcript variant 1, mRNA [NM_000856] | 10.0507765 |
| H19 | Homo sapiens H19, imprinted maternally expressed transcript (non-protein coding) (H19), non-coding RNA | 10.613719 |
| | [NR_002196] | |
| HPSE2 | Homo sapiens heparanase 2 (HPSE2), transcript variant 1, mRNA [NM_021828] | 5.770388 |
| HTATSF1P2 | Homo sapiens cDNA FLJ46534 fis, clone THYMU3037052, weakly similar to Homo sapiens HIV TAT | 43.155224 |
| | specific factor 1 (HTATSF1), [AK128391] | |
| HTR4 | Homo sapiens 5-hydroxytryptamine (serotonin) receptor 4 (HTR4), transcript variant d, mRNA | 5.7528954 |
| | [NM_001040172] | |
| IGSF11 | Homo sapiens immunoglobulin superfamily, member 11 (IGSF11) transcript variant 1, mRNA [NM_152538] | 21.946852 |
| IQCA1 | Homo sapiens IQ motif containing with AAA domain 1 (IQCAI), mRNA [NM_024726] | 8.559639 |
| ITM2A | Homo sapiens integral membrane protein 2A (ITM2A), transcript variant 1, mRNA [NM_004867] | 9.097328 |
| KRTEID12 | Homo sapiens kelch repeat and RTR (PO7) domain containing 12 (KRTRD12), mRNA [NM_707335] | 8.086248 |
| KCND1 | Homo sapiens potassium voltage-gated channel, Shal-related subfamily, member 1 (KCND1), mRNA | 8.62409 |
| | [NM_004979] | |
| KCNQ1 | Homo sapiens potassium voltage-gated channel, NOT-like subfamily, member 1 (KCNQ1), transcript variant | 8.957807 |
| | 1, mRNA [NM_000218] | |
| KIAA0226L | Homo sapiens chromosome 13 open reading frame 18, mRNA (cDNA clone IMAGE: 5212065), [BC032311] | 15.101533 |
| KIAA1244 | Homo sapiens KIAA1244 (KIAA1244), mRNA [NM_020340] | 6.4185023 |
| KIAA1244 | Homo sapiens KIAA1244 (KIAA1244), mRNA [NM_020340] | 10.0645685 |
| KRT16P3 | Homo sapiens keratin 16 pseudogene 3 (KRT16P3), non-coding RNA [NR_029393] | 7.0826035 |
| LINC00301 | Homo sapiens long intergenic non-protein coding RNA 301 (LINC00301), non-coding RNA [NR_026946] | 5.88876 |
| LINC00309 | Homo sapiens long intergenic non-protein coding RNA 305 (LINC00309), non-coding RNA [NR_033837] | 8.750379 |
| LINC00477 | Homo sapiens long intergenic non-protein coding RNA 477 (LINC00477), non-coding RNA [NR_029451] | 7.6955123 |
| LMF1 | Homo sapiens lipase maturation factor 1 (LMF1), transcript variant 4, non-coding RNA [NR_036442] | 59.499176 |
| LOC100129198 | Homo sapiens clone FLC0664 PRO2866 mRNA, complete cds [AF130117] | 509.58444 |
| LOC100131138 | Homo sapiens uncharacterized LOC100131138 (LOC100131138), non-coding RNA [NR_036513] | 13.540075 |
| LOC100134091 | Homo sapiens cDNA FLJ45377 fis, clone BRHIP3019956, [AK127309] | 5.8445344 |
| LOC100233156 | Homo sapiens tektin 4 pseudogene (LOC100233156), transcript variant 1, non-coding, RNA [NR_037871] | 12.5568075 |
| LOC100506388 | Homo sapiens uncharacterized LOC100506388 (LOC100506388), transcript variant 1, mRNA | 9.898991 |
| | [NM_001242780] | |
| LOC157860 | Homo sapiens cDNA: FLJ22090 fis, clone HEP16084, [AK025743] | 33.94284 |
| LOC 158696 | Homo sapiens uncharacterized LOC158696 (LOC158696), non-coding RNA [NR_026935] | 5.652232 |
| LOC283665 | Homo sapiens hypothetical protein LOC283665, mRNA (cDNA clone IMAGE: 4826990), [BC034958] | 94.39905 |
| LOC388630 | Homo sapiens UPF0632 protein A (LOC386630), mRNA [NM_001194986] | 44.88917 |
| LOC400752 | Homo sapiens uncharacterized LOC400752 (LOC400752), non-coding RNA [NR_024270] | 7.1026893 |
| LRRC70 | Homo sapiens leucine rich repeat containing 70 (LRRC70), mRNA [NM_181506] | 7.3285074 |
| LRRN1 | Homo sapiens leucine rich repeat neuronal 1 (LRRN1), mRNA [NM_020873] | 7.5075636 |
| MAB21L2 | Homo sapiens mab-21-like 2 (C. elegans) (MAB21L2), mRNA [NM_006439] | 5.443071 |
| MAML3 | Homo sapiens mastermind-like 3 (Drosophila) (MAML3), mRNA [NM_018717] | 21.73409 |
| MECOM | Human MDS16 (MDS1) mRNA, complete cds, [U43292] | 23.16021 |
| MIR133A1 | Homo sapiens microRNA 133a-1 (MIR133A1), microRNA [NR_029675] | 7.9685173 |
| MLIP | Homo sapiens muscular LMNA-interacting protein (MLIP), mRNA [NM_138569] | 5.463037 |
| MLL3 | Homo sapiens myeloid/lymphoid or mixed-lineage leukemia 3 (MLL3), mRNA [NM_170606] | 6.755415 |
| MYO1G | myosin IG [Source: HGNC Symbol: Acc: 13880] [ENST00000480503] | 36.72139 |
| NAIP | Homo sapiens NLR family, apoptosis inhibitory protein (NAIP), transcript variant 1, mRNA [NM_004536] | 5.0188084 |
| NAIP | Homo sapiens NLR family, apoptosis inhibitory protein (NAIP), transcript variant 1, mRNA [NM_094536] | 14.485689 |
| NAIP | Homo sapiens NLR family, apoptosis inhibitory protein (NAIP), transcript variant 1, mRNA [NM_004536] | 26.855295 |
| NCKAP5 | Homo sapiens cDNA FLJ34870 fis, clone NT2NE2014651, [AK092189] | 5.0321865 |
| NKAIN4 | Homo sapiens Na+/K+ transporting ATPase interacting 4 (NKAIN4), mRNA [NM_152864] | 5.4856596 |
| NR0B7 | Homo sapiens nuclear receptor subfamily 0, group R, member 2 (NR0R2), mRNA [NM_071969] | 16.071416 |
| NRIP2 | Homo sapiens nuclear receptor interacting protein 2 (NRIP2) mRNA [NM_031474] | 13.104872 |
| NRXN3 | Homo sapiens neurexin 3 (NRXN3), transcript variant 1, mRNA [NM_004796] | 5.961855 |
| OR2T8 | Homo sapiens olfactory receptor, family 2, subfamily T, member 8 (OR2T8), mRNA [NM_001005522] | 37.168884 |
| OTOP3 | Homo sapiens otopetrin 3 (OTOP3), mRNA [NM_178233] | 17.833977 |
| P2RY13 | Homo sapiens purinergic receptor P2Y, G-protein coupled, 13 (P2RY13), mRNA [NM_176894] | 10.975073 |
| PDIA2 | Homo sapiens protein disulfide isomerase family A, member 2 (PDIA2), mRNA [NM_006849] | 343.55597 |
| PECAM1 | Homo sapiens platelet/endothelial cell adhesion molecule (PECAM1), mRNA [NM_000442] | 12.3957815 |
| PIK3R6 | Homo sapiens phosphoinositide-3-kinase, regulatory subunit 6 (PIK3R6), mRNA [NM_001010855] | 8.433382 |
| POTEB | Homo sapiens POTE ankyrin domain family, member B (POTED), mRNA [NM_207355] | 10.403516 |
| POTED | Homo sapiens POTE ankyrin domain family, member D (POTED), mRNA [NM_174981] | 6.567783 |
| PRR15 | Homo sapiens proline rich 15 (PRR15), mRNA [NM_175887] | 9.117226 |
| RIIAD1 | Homo sapiens regulatory subunit of type II PKA R-subunit (RII8) domain containirg 1 (RIIAD1), mRNA | 47.810894 |
| | [NM_001144956] | |
| SAMD10 | Homo sapiens sterile alpha motif domain containing 10 (SAMD10), mRNA [NM_080621] | 5.4775047 |
| SCRG1 | Homo sapiens stimulator of chondrogenesis 1 (SCRG1), mRNA [NM_007281] | 8.318421 |
| SERPINF2 | Homo sapiens serpin peptidase inhibitor, clade F (alpha-2 antiplasmin, pigment epthelium derived factor), | 15.328115 |
| | member 2 (SERPINF2), transcript variant 1, mRNA [NM_000934] | |
| SHOX2 | Homo sapiens short stature homeobox 2 (SHOX2), transcript variant 2, mRNA [NM_006584] | 36.967613 |
| SIK1 | Homo sapiens salt-inducible kinase 1 (SIK1), mRNA [NM_173354] | 9.342587 |
| SIK3 | SIK family kinase 3 [Source: HGNC Symbol; Acc: 29165] [ENST00000480463] | 8.160866 |
| SLC2A2 | Homo sapiens solute carrier family 2 (facilitated glucose transporter), member 2 (SLC2A2), mRNA | 45.076607 |
| | [NM_000340] | |
| SLC34A2 | Homo sapiens solute carrier family 34 (sodium phosphate), member 2 (SLC34A2), transcript variant 1, | 14.32704 |
| | mRNA [NM_006424] | |
| SLC35F4 | Homo sapiens solute carrier family 35, member F4 (SLC35F4), mRNA [NM_001206920] | 127.62837 |
| SLC7A4 | Homo sapiens solute carrier family 7 (orphan transporter), member 4 (SLC7A4), mRNA [NM_004173] | 5.285855 |
| SLC9B1 | Homo sapiens solute carrier amily 9, subfamily B (cation proton antiporter 2), member 1 (SLC9B1), nuclear | 24.522345 |
| | gene encoding mitochondrial protein transcript variant 1, mRNA [NM_139173] | |
| SLCO4C1 | Homo sapiens solute carrier organic anion transporter family, member 4C1 (SLCO4C1), mRNA | 5.383931 |
| | [NM_180991] | |
| SLITRK2 | Homo sapiens SLIT and NTRK-like family member 2 (SLITRK2) transcript variant 1, mRNA [NM_032539] | 8.09339 |
| SOHLH2 | Homo sapiens spermatogenesis and oogenesis specific basic helix-loop-helix 2 (SOHLH2), mRNA | 6.3827267 |
| | [NM_017826] | |
| SPIB | Homo sapiens Spi-B transcription factor (Spi-1/PU.1 related) (SPIB), transcript variant 1, mRNA | 6.160538 |
| | [NM_003121] | |
| SQSTM1 | Human phosphotyrosine independent ligand p62B B-cell isoform for the Lck SH2 domain mRNA, partial | 21.140625 |
| | cds, [U46752] | |
| STON1- | Homo sapiens STON1-GTF2A1L readthrough (STON1-GTF2A1L), transcript variant 1, mRNA | 22.73415 |
| GTF2A1L | [NM_172311] | |
| STYK1 | Homo sapiens serine/threonine/tyrosine kinase 1 (STYK1), mRNA [NM_018423] | 6.1324606 |
| SULT1C2 | Homo sapiens sulfotransferase family, cytosolic, 1C, member 2 (SULT1C2), transcript variant 2, mRNA | 5.3968763 |
| | [NM_176825] | |
| SULT1C4 | Homo sapiens sulfotransferase family, cytosolic, 1C, member 4 (SULT1C4), mRNA [NM_006588] | 112.268 |
| SYNPO2L | Homo sapiens synaptopodin 2-like (SYNPO2L), transcript variant 2, mRNA [NM_024875] | 9.352724 |
| SYTL1 | Homo sapiens synaptotagmin-like 1 (SYTL1), transcript variant 2, mRNA [NM_032872] | 6.56714 |
| TBKBP1 | Homo sapiens TBK1 binding protein 1 (TBKBP1), mRNA [NM_014726] | 5.8236227 |
| TEKT4 | Homo sapiens tektin 4 (TEKT4), mRNA [NM_144705] | 9.601399 |
| TEKT4P2 | Homo sapiens tektin 4 pseudogene 2 (TEKT4P21, transcript variant 3, non-coding RNA [NR_038329] | 9.088646 |
| TET1 | Homo sapiens tet methylcytosine dioxygenase 1 (TET1), mRNA [NM_030625] | 15.013668 |
| THNSL2 | Homo sapiens threonine synthase-like 2 (S. cerevisiae) (THNSL2), transcript variant 1, mRNA | 5.326104 |
| | [NM_018271] | |
| TINAG | Homo sapiens tubulointerstitial nephritis antigen (TINAG), mRNA [NM_014464] | 29.155573 |
| TMEM132D | Homo sapiens transmembrane protein 132D (TMEM132D), mRNA [NM_133448] | 7.51078 |
| TMEM2 | Homo sapiens transmembrane protein 2 (TMEM2), transcript variant 1, mRNA [NM_013390] | 8.420662 |
| TMEM31 | Homo sapiens transmembrane protein 31 (TMEM31), mRNA [NM_182541] | 7.3745914 |
| TMSB4Y | Homo sapiens hymosin beta 4, Y-linked (TMSB4Y), mRNA [NM_004202] | 16.171171 |
| TPD52 | Homo sapiens tumor protein D52 (TPD52), transcript variant 1, mRNA [NM_001025252] | 11.895537 |
| TPD52 | Homo sapiens tumor protein D52 (TPD52), transcript variant 1, mRNA [NM_001025252] | 17.488007 |
| TPPP2 | Homo sapiens tubule polymerization-promoting protein family member 2 (TPPP2), mRNA [NM_173846] | 10.157727 |
| TPTE2P6 | Homo sapiens transmembrane phosphoinositide 3-phosphatase and tensin homolog 2 pseudogene 6 | 12.2382765 |
| | (TPTE2P6), non-coding RNA [NR_002815] | |
| TRAT1 | Homo sapiens T cell receptor associated transmembrane adaptor 1 (TRAT1), mRNA [NM_016388] | 9.758402 |
| TREH | Homo sapiens trehalase (brush-border membrane glycoprotein) (TREH), mRNA [NM_007180] | 6.175398 |
| TRIM36 | Homo sapiens tripartite motif containing 36 (TRIM36), transcript variant 1, mRNA [NM_018700] | 6.563853 |
| TRPM1 | Homo sapiens transient receptor potential cation channel, subfamily M, member 1 (TRPM1) transcript | 7.071682 |
| | variant 2, mRNA [NM_002420] | |
| TUSC3 | Homo sapiens cDNA: FLJ22496 fis, clone HRC11236, [AK026149] | 9.696791 |
| USE2QL1 | Homo sapiens ubiquitin-conjugating enzyme E2Q family-like 1 (UBE2QL1), mRNA [NM_001145161] | 7.371957 |
| UBR4 | ubiquitin protein ligase E3 component n-recognin 4 [Source: HGNC Symbol; Acc: 30313] | 8.200464 |
| | [ENST00000419533] | |
| VP553 | Homo sapiens vacuolar protein sorting 53 homolog (S. cerevisiae) (VP553), transcript variant 2, mRNA | 16.601204 |
| | [NM_018289] | |
| ZBTB32 | Homo sapiens zinc finger and BTB domain containing 32 (ZBTB32), mRNA [NM_014383] | 9.604526 |
| ZCCHCS | Homo sapiens zinc finger, CCHC domain containing 5 (ZCCHC5), mRNA [NM_152694] | 5.275995 |
| ZNF423 | Homo sapiens zinc finger protein 423 (ZNF423), mRNA [NM_015069] | 7.5440564 |

The dental pulp stem cell to be used in producing a graft material for treating nerve damage according to the specification may be a dental pulp stem cell in which the expression levels of one type or more, two types or more, 3 types or more, 4 types or more, 5 types or more, 6 types or more, 7 types or more, 8 types or more, 9 types or more, 10 types or more, 11 types or more, 12 types or more, 13 types or more or 14 types of genes selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2, and HTATSF1P2, each are 5 times or more, 10 times or more, 20 times or more, 30 times or more, 40 times or more, 50 times or more, 60 times or more, 70 times or more, 80 times or more, 90 times or more, 95 times or more, or 100 times or more as low as an average expression level in the dental pulp stem cells.

Among two types or more groups of dental pulp stem cells, a group of dental pulp stem cells in which the gene expression level of the group of genes listed in Table 2 is low, may be selected and used as the dental pulp stem cell to be used in producing a graft material for treating nerve damage according to the specification.

More specifically, a group of dental pulp stem cells in which the expression levels of the genes of 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 98% or more of the group of genes described in Table 2, each are 5 times or more, 10 times or more, 20 times or more, 30 times or more, 40 times or more, 50 times or more, 60 times or more, 70 times or more, 80 times or more, 90 times or more, 95 times or more, or 100 times or more as low as other groups of cells, may be selected and used.

Among two types or more groups of dental pulp stem cells, a group of dental pulp stem cells in which the expression levels of one type or more, two types or more, 3 types or more, 4 types or more, 5 types or more, 6 types or more, 7 types or more, 8 types or more, 9 types or more, 10 types or more, 11 types or more, 12 types or more, 13 types or more or 14 types of genes selected from the group consisting of Gafa3, Lmf1, Fam13a, Gkn1, Gpr112, Sultic4, Slc35f4, Gstt1, Gpr27, Pdia2, RIIAD1, GSTT1, SLC2A2 and HTATSF1P2, each are 5 times or more, 10 times or more, 20 times or more, 30 times or more, 40 times or more, 50 times or more, 60 times or more, 70 times or more, 80 times or more, 90 times or more, 95 times or more, or 100 times or more as low as other groups of cells, may be selected and used as the dental pulp stem cell to be used in producing a graft material for treating nerve damage according to the specification.

The effect of the graft material for treatment obtained by the method for producing a graft material for treating nerve damage according to the present invention can be evaluated by the following method using, for example, a disease-model animal.

A rat middle cerebral artery occlusion (MCAO) model can be prepared by a known method and more specifically prepared as follows. A wild type rat was anesthetized. While maintaining the rectal temperature at 37±0.5° C. under anesthesia, the cervical region was dissected to expose a branched part of the right carotid artery and internal and external carotid arteries were separated. Thereafter, a 4-0 nylon thin thread having a tip rounded off by silicon coat was inserted from the external carotid artery, allowed to reach the beginning of the middle cerebral artery through the internal carotid artery and fixed there. In this manner, blood flow in the right side middle cerebral artery region was blocked to cause ischemia. After the ischemia state was maintained for one hour, the nylon thin thread was withdrawn out of the middle cerebral artery to allow perfusion to start again.

At the 48th hour after the perfusion was started again, an effective amount of graft material (dental pulp stem cell) for treatment according to the present invention was administered from the caudal vein. Alternatively, the effective amount of graft material (dental pulp stem cell) for treatment may be locally administered to an infarction site.

Recovery of motor function is evaluated based on BBB score (Basso D M et al., J Neurotrauma. 1995 February; 12 (1): 1-21).

### [Graft Material for Treating Nerve Damage]

The graft material for treating nerve damage according to the present invention is produced by the method for producing a graft material for treating nerve damage according to the present invention described above and contains a dental pulp stem cell and a medium substantially containing no growth factors except FGF2. The “dental pulp stem cell” and the “medium substantially containing no growth factors except FGF2” are the same as defined above. The graft material may contain gel such as collagen gel, soft agar and a synthetic polymer and the viscosity may be controlled by an appropriate gelation agent or a thickening agent.

The dental pulp stem cell to be used in a graft material for treating nerve damage according to the specification may be a dental pulp stem cell in which the gene expression level of the group of genes listed in Table 1 is high compared to the average expression level of genes in dental pulp stem cells. The “dental pulp stem cell, in which the gene expression level of the group of genes listed in Table 1 is high compared to the average expression level of genes in dental pulp stem cells” is the same as described above.

The graft material for treating nerve damage according to the specification may be a graft material using a dental pulp stem cell in which compared to an average expression level in the dental pulp stem cells, the expression levels of one type or more, two types or more, 3 types or more, 4 types or more, 5 types or more, 6 types or more, 7 types or more, 8 types or more, 9 types or more, 10 types or more, 11 types or more, 12 types or more, 13 types or more, 14 types or more, 15 types or more, 16 types or more, 17 types or more, 18 types or more, 19 types or more, 20 types or more, or 21 types of genes selected from the group consisting of MYO1G, RBMY2FP, FILIP1, Clorf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg, and Cbln2, each are 5 times or more, 10 times or more, 20 times or more, 30 times or more, 40 times or more, 50 times or more, 60 times or more, 70 times or more, 80 times or more, 90 times or more, 95 times or more, or 100 times or more as high as other groups of cells.

As the dental pulp stem cell to be used in the graft material for treating nerve damage according to the specification, a group of dental pulp stem cells in which the gene expression level of the group of genes listed in Table 1 is high may be selected and used from two types or more groups of dental pulp stem cells and used. The “dental pulp stem cells in which the gene expression level of the group of genes listed in Table 1 is high, may be selected and used from two types or more groups of dental pulp stem cells” is the same as defined above.

Among two types or more groups of dental pulp stem cells, a group of dental pulp stem cells in which the expression levels of one type or more, two types or more, 3 types or more, 4 types or more, 5 types or more, 6 types or more, 7 types or more, 8 types or more, 9 types or more, 10 types or more, 11 types or more, 12 types or more, 13 types or more, 14 types or more, 15 types or more, 16 types or more, 17 types or more, 18 types or more, 19 types or more, 20 types or more, or 21 types of genes selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg, and Cbln2, each are 5 times or more, 10 times or more, 20 times or more, 30 times or more, 40 times or more, 50 times or more, 60 times or more, 70 times or more, 80 times or more, 90 times or more, 95 times or more, or 100 times or more as high as other groups of cells, may be selected and used as the dental pulp stem cell to be used in the graft material for treating nerve damage according to the specification.

The dental pulp stem cell to be used in the graft material for treating nerve damage according to the specification may be a dental pulp stem cell, in which the gene expression level of the group of genes listed in Table 2 is low compared to an average expression level in dental pulp stem cells.

The “dental pulp stem cell, in which the gene expression level of the group of genes listed in Table 2 is low compared to an average expression level in dental pulp stem cells” is the same as described above.

The dental pulp stem cell to be used in the graft material for treating nerve damage according to the specification may be a dental pulp stem cell, in which the expression levels of one type or more, two types or more, 3 types or more, 4 types or more, 5 types or more, 6 types or more, 7 types or more, 8 types or more, 9 types or more, 10 types or more, 11 types or more, 12 types or more, 13 types or more or 14 types of genes selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2 and HTATSF1P2, each are 5 times or more, 10 times or more, 20 times or more, 30 times or more, 40 times or more, 50 times or more, 60 times or more, 70 times or more, 80 times or more, 90 times or more, 95 times or more, or 100 times or more as low as an average expression level in the dental pulp stem cells.

The dental pulp step cell to be used in the graft material for treating nerve damage according to the specification includes a graft material using a dental pulp stem cell in which the gene expression level of the group of genes listed in Table 2 is low, of two types or more groups of dental pulp stem cells.

The “dental pulp stem cell, in which the gene expression level of the group of genes listed in Table 2 is low, of two types or more groups of dental pulp stem cells” is the same as described above.

Among two types or more groups of dental pulp stem cells, a group of dental pulp stem cells, in which the expression levels of one type or more, two types or more, 3 types or more, 4 types or more, 5 types or more, 6 types or more, 7 types or more, 8 types or more, 9 types or more, 10 types or more, 11 types or more, 12 types or more, 13 types or more or 14 types of genes selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2 and HTATSF1P2, each are 5 times or more, 10 times or more, 20 times or more, 30 times or more, 40 times or more, 50 times or more, 60 times or more, 70 times or more, 80 times or more, 90 times or more, 95 times or more, or 100 times or more as low as other groups of cells, may be selected and used as the dental pulp stem cell to be used in a graft material for treating nerve damage according to the specification.

[Method for Treating Nerve Damage]

The present invention includes a method for treating nerve damage, including a step of grafting the graft material for treating nerve damage as mentioned above to an area of nerve damage.

The graft material for treating nerve damage can be injected into an area of nerve damage by e.g., a syringe. Alternatively, the area of nerve damage is dissected and then the graft material may be disposed. In the case where the graft material contains a xenogeneic cell, an immune suppressant such as cyclosporine can be administered together. As long as a nerve damage therapeutic effect can be obtained, the grafting material can be used in combination with other medicinal drugs.

The dose and administration times can be appropriately determined by those skilled in the art.

The subject to which the method for treating a nerve damage is to be applied is not limited to humans and may be other mammals (for example, mice, rats, rabbits, dogs, cats, monkeys, sheep, cows, horses).

In the specification, an example of a method for treating human brain infarction will be described below; however, the treatment method is not limited to the following example.

The graft material for treatment produced by the method for producing a graft material for treating nerve damage according to the present invention or the graft material for treating nerve damage according to the present invention, more specifically, dental pulp stem cells, are intravenously administered by use of a syringe pump from a peripheral vein at an injection rate of 2 mL/minute to a patient with human brain infarction, in an effective amount.

### [Kit for Producing Graft Material for Treating Nerve Damage]

The present invention includes a kit for producing a graft material for treating nerve damage. The kit contains a medium for culturing a dental pulp stem cell or all or part of components of the medium and FGF2. As the medium for culturing a dental pulp stem cell, a base medium or a medium for culturing a mesenchymal stem cell is mentioned. FGF2 and the medium may be separately contained or may be mixed together from the beginning. Furthermore, under the assumption that ultra-pure water, which is a material regularly stocked in laboratories, can be prepared by the user, all or part of requisite components for a medium may be contained so as to prepare the medium of the present invention only by adding the components to the water.

The kit of the present invention may be used in experiments performed in laboratories or used in a large scale culture. The kit may contain, other than a culture solution, e.g., a culture container, a virus filter, a coating material for a culture container, various reagents, a buffer and an instruction booklet.

The disclosures of all Patent Literatures and Non Patent Literatures cited in the specification are incorporated herein in their entirety by reference.

### Examples

Now, the present invention will be more specifically described based on Examples; however, the present invention is not limited to these. Those skilled in the art can modify the invention in various ways without departure from the significance of the present invention and such modifications are included in the range of the present invention.

### Example 1

### Effect 1 of Difference in Culture Method of Dental Pulp Stem Cell Upon Motor Function Recovery Effect of Model with Total Amputation of Spinal Cord

1. Experimental Method

1-1. Animal and Material

Wistar rats (7 weeks old, female) were purchased from Japan SLC and an anesthetic drug, somnopentyl, was purchased from Kyoritsuseiyaku Corporation. Prior to animal experiments, a protocol of animal experiment was prepared in accordance with a predetermined format based on the regulation for safety and welfare of animal experiments and approval by the animal breeding/animal experiment committee of Gifu Pharmaceutical University was obtained.

1-2. Cell Culture

From the evulsion tooth excised out, dental pulp stem cells were induced and proliferated in culture in accordance with the previous report (Tamaoki et al., J Dent Res. 2010 89: 773-778). The dental pulp stem cells successively cultured up to the 8th generation were sub-cultured in MSCGM medium (LONZA) 2 to 5 times to prepare cells (DP310) and sub-cultured in a-MEM medium (Sigma) containing 10 ng/mL FGF2 and 10% FCS, 5 or 6 times to prepare dental pulp stem cells (DP31F).

1-3. Experimental Method using Total Amputation Model

Preparation of Model with Total Amputation of Spinal Cord

To Wistar rats (7 weeks old, female), somnopentyl was intraperitoneally administered in a dose of 40 mg/kg body weight. After anesthesia, the back was dissected along the midline at the position of the 10th thoracic spine in a length of 2 cm. The fat and muscle tissues were removed to expose the spine. The vertebral arch was removed and the 10th thoracic spine (T10) was dissected cross-sectionally with a sharp knife. After arrest of bleeding, the cultured dental pulp stem cells, which were suspended in each medium so as to contain 10⁶ cells/10 μL, were injected to the space between the rostral cut-end and the caudal cut-end of the cleavage site. Thereafter, the muscles of the back and the skin were sutured. After the surgery, it was confirmed that the hind limb at the same side of the cleaved spinal cord was paralyzed. The rats were raised in a routine manner and subjected to experiments. Note that cyclosporine serving as an immune suppressant was intraperitoneally injected in a dose of 10 mg/kg, every day.

2. Results

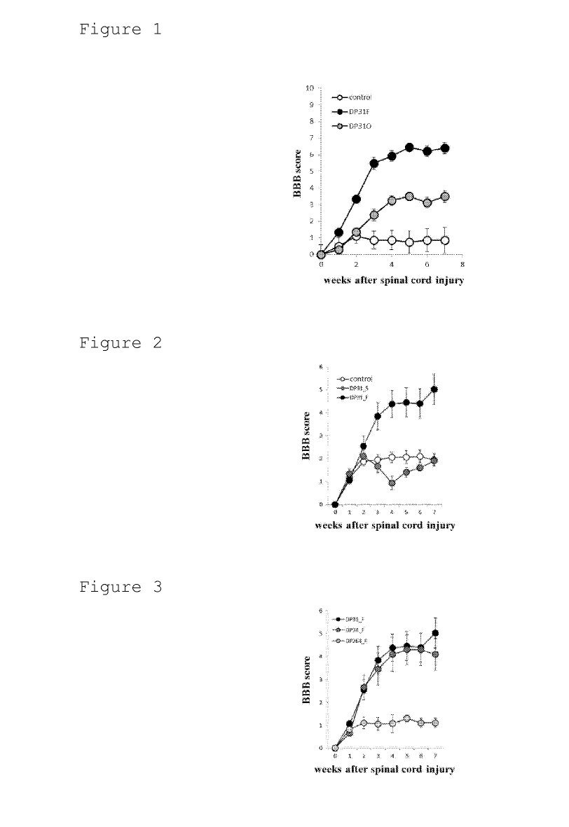

Recovery of motor function was evaluated based on BBB score (Basso D M et al., J Neurotrauma. 1995 February; 12 (1): 1-21) (FIG. 1).

Two weeks later, even an individual to a damaged part of which the cells were not injected but PBS or a culture supernatant alone was injected, recovered to the extent that one or two joints of the hind limb completely paralyzed slightly moved. However, no more recovery of motor function was observed in 7 weeks after the damage (BBB score=1).

In contrast, in the group having DP310 grafted, as shown in FIG. 1, three weeks after the damage, two joints became sufficiently movable in a half number of the individuals (7 out of 14). Four weeks after the damage, all individuals of the group showed significantly high motor function compared to the control group (BBB score=3.5). In the group having DP31F grafted, one week after the damage, one joint of the hind limb became slightly movable. On and after two weeks, the individuals showed significantly high motor function compared to the control group and the DP310 grafted group (final BBB score=6.5). The half of them (7 out of 13) was recovered to the extent that the body weight was supported by the paralyzed limb.

### Example 2

### Effect 2 of Difference in Culture Method of Dental Pulp Stem Cell Upon Motor Function Recovery Effect of Model with Total Amputation of Spinal Cord

1. Experimental Method

1-1. Animal and Material

Animals were prepared in the same manner as in Example 1.

1-2. Cell Culture

From the evulsion tooth excised out, dental pulp stem cells were induced and proliferated in culture in accordance with the previous report (Tamaoki et al., J Dent Res. 2010 89: 773-778). The dental pulp stem cells successively cultured up to the 12nd generation in MSCBM medium (LONZA) were sub-cultured in α-MEM medium (Sigma) containing 10% FCS, 7 or 8 times to prepare dental pulp stem cells (DP31S) and sub-cultured in α-MEM medium containing 10 ng/mL FGF2 and 10% FCS, 7 or 8 times to prepare dental pulp stem cells (DP31F).

1-3. Experimental method using total amputation model Models with total amputation of spinal cord was prepared in the same manner as in Example 1.

2. Results

In the same manner as in Example 1, recovery of motor function was evaluated based on BBB score (Basso D M et al., J Neurotrauma. 1995 February; 12 (1): 1-21) (FIG. 2).

In the group (control) where cells were not grafted and the group where dental pulp stem cells (DP31S) cultured in FGF2 free α-MEM medium containing 10% FSC were grafted, motor function recovery of the hind limb was rarely observed. Only one joint, in average, was slightly movable (BBS score=1.9±0.2, n=45, BBS score=1.9±0.2, n=14, respectively).

In contrast, in the group where dental pulp stem cells (DP31F) cultured in FGF2 containing a-MEM medium containing 10% FSC, were grafted, significant recovery effect of motor function was observed. In average, all three joints became movable (BBS score=5.0±0.7, n=28).

### Example 3

### Effect of Difference in Donor of Dental Pulp Stem Cell Upon Motor Function Recovery Effect of Model with Total Amputation of Spinal Cord

1. Experimental Method

1-1. Animal and Material

Animals were prepared in the same manner as in Example 1.

1-2. Cell Culture

From evulsion teeth excised out from three different donors (DP31, DP74, and DP264), dental pulp stem cells were induced, and proliferated in culture in accordance with the previous report (Tamaoki et al., J Dent Res. 2010 89: 773-778). The dental pulp stem cells derived from three donors each successively cultured 7 or 8 times in α-MEM medium containing 10 ng/mL FGF2 and 10% FCS to prepare dental pulp stem cells (DP31F, DP74F, and DP264F) derived from three donors.

1-3. Experimental method using total amputation model In Models with total amputation of spinal cord were prepared in the same manner as in Example 1.

2. Results

Recovery of motor function was evaluated based on BBB score (Basso D M et al., J Neurotrauma. 1995 February; 12 (1): 1-21) in the same manner as in Example 1 (FIG. 3).

In the DP264F grafted group, compared to DP31F and DP74F grafted groups (BBB score=4.1±0.7, n=12), no recovery effect of motor function was observed (BBB score=1.1±0.2, n=14).

### Experimental Example 1

### Effect of Difference in Culture Method of Dental Pulp Stem Cell Upon Expression of Differentiation Marker of Nervous System Cell

1. Experimental Method

The rats of Example 1 in which dental pulp stem cells DP310 were grafted and the rats of Example 2 in which dental pulp stem cells DP31F were grafted, each were subjected to transcardial perfusion fixation with 0.1 M phosphate buffer (pH7.3) containing 4% paraformaldehyde, 7 weeks after grafting and a spinal cord tissue was excised out.

The spinal cord tissue excised out was soaked in a 20% sucrose solution in accordance with a conventional method, embedded in an OCT compound and sliced into thin sections by a cryostat. The thin sections were attached on slide glasses, soaked in Tris-HCl (pH7.4) containing 0.3% Triron X100 (registered trade mark) to enhance cell membrane permeability with an antibody, blocked with PBS containing 2% blockace (DS Pharma Biomedical Co., Ltd.) at room temperature for 30 minutes. Immunostaining was performed with a primary antibody such as anti-Human Nuclear antigen antibody (Millipore (MAB1281)), anti-Tujl antibody (Cell Signaling technology, #5568), anti-myelin basic protein (MBP) antibody (Millipore (AB980)), anti-CNPase antibody (Sigma (C5922)), anti-glial fibrillary acidic protein (GFAP) antibody (Dako (Z0334)), anti-growth asscciated protein 43 (GAP43) antibody (Chemicon (MAB347)) or anti-green fluorescence protein (GFP) antibody (Chemicon (AB3080)).

Human Nuclear antigen is a marker for human cells; CNPase is a marker for immature oligodendrocytes; and GFAP is a marker for immature astrocyte. Note that the graft cells were designed to express a GFP gene by use of a retroviral vector.

2. Results

The grafted dental pulp stem cells were identified based on GFP or Human Nuclear antigen positive. In DP310 before grafting, all markers (more specifically, Tuj1 (immature nerve cell marker), GFAP, CNPase, and Nestin (stem cell marker)) observed in immature nervous system cells, expressed; whereas, in DP310 in the spinal cord tissue, almost all cells were negative to Tuj1 and GFAP and positive to CNPase. In contrast in DP31F in the spinal cord tissue, a predetermined ratio of cells were positive to Tuj1 and MBP and almost all cells were negative to GFAP (it was not confirmed whether the cells positive to Tuj1 and positive to MBP are the same or not).

Thus, the possibility that grafted DP310 may be differentiated into oligodendrocytes and DP31F into a cell population containing nerve cells and oligodendrocytes, was suggested.

From the above, it was suggested that the following features were added to the dental pulp stem cells treated with FGF2, more specifically, to the cells cultured in MSCGM.

(i) When grafted in damaged spinal cord, the cells are changed into cells specifically differentiated into nerve cells.

(ii) Differentiation potency is limited (more specifically, the cells are specifically differentiated into nerve cells); however, proliferation potency is maintained.

These features indicate that the dental pulp stem cells treated with FGF2 are useful for treating nerve damage.

### Experimental Example 2

### Global Gene Expression Analysis

1. Experimental Method

The dental pulp stem cells derived from two donors (DP31, and DP264) were cultured in MSCBM medium. Total RNA was extracted from the above dental pulp stem cells by RNeasy Plus Mini kit (Qiagen). After the RNA was quantified by Agilent 2100 Bioanalyzer (Agilent Technologies), 250 mg of RNA was taken and subjected to reverse transcription to obtain cDNA, which was amplified and labeled with Cy3-labeled CTP, by use of Low Input Quick Amp Labeling kit (Agilent Technologies) in accordance with the instruction booklet attached thereto. After the cDNA was purified, the cDNA was quantified by use of ND-1000 Spectrophotometer (Nano Drop Technologies) and allowed to hybridize to Whole Human Genome 4×44K oligo-DNA microarray (Agilent Technologies). After the hybridization, the array was continuously washed with Gene Expression Wash Pack (Agilent Technologies). The fluorescent image of the hybridized array was prepared by Agilent DNA Microarray Scanner (Agilent Technologies) and the fluorescent intensity was analyzed by Agilent Feature Extraction software ver.10.7.3.1. (Agilent Technologies). Analysis was made once with respect to each sample. The level of gene expression was analyzed by Gene Spring GX11.5 (Agilent Technologies).

2. Results

The genes of DP264 whose expression levels were 5 times or more as high as those in DP31 were listed in Table 1 and the genes of DP264 whose expression levels were 5 times or more as low as those in DP31 were listed in Table 2.

1-27. (canceled)

28. A method for producing a graft material for treating nerve damage, comprising
a step of culturing a dental pulp stem cell in a medium substantially containing no growth factors except FGF2, wherein
the dental pulp stem cell used is a dental pulp stem cell in which
(i) the expression level of 10% or more of genes in the group of genes listed in Table 1 is 5 times or more as high as an average expression level of the genes in dental pulp stem cells,
(ii) the expression level of at least one gene selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg and Cbln2 is 5 times or more as high as an average expression level of the genes in dental pulp stem cells,
(iii) the expression level of 10% or more of genes in the group of genes listed in Table 2 is 5 times or more as low as an average expression level of the genes in dental pulp stem cells, or
(iv) the expression level of at least one gene selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2, and HTATSF1P2 is 5 times or more as low as an average expression level of the genes in dental pulp stem cells.

29. The method according to claim 28, wherein the average expression level of the genes in dental pulp stem cells is calculated based on expression levels of two or more groups of dental pulp stem cells.

30. The method according to claim 28, wherein the medium substantially containing no growth factors except FGF2 is a serum-containing base medium supplemented with FGF2 alone as a growth factor.

31. The method according to claim 30, wherein the serum in the medium has a concentration of less than 15 wt %.

32. The method according to claim 28, wherein the medium substantially containing no growth factors except FGF2 is a commercially available medium for culturing mesenchymal stem cells supplemented with FGF2 alone as a growth factor.

33. The method according to claim 28, wherein FGF2 in the medium has a concentration of 5 ng/mL or more, or 7 ng/mL or more.

34. The method according to claim 28, wherein the nerve damage is spinal cord injury, cerebral infarction, intracerebral hemorrhage, subarachnoid hemorrhage, spinal hemorrhage, compression injury of nerve caused by disk herniation, sciatic nerve pain or peripheral nerve damage caused by diabetes.

35. A graft material for treating nerve damage, wherein the graft material is produced by the method according to claim 28.

36. The graft material for treating nerve damage according to claim 35, wherein the medium substantially containing no growth factors except FGF2 is a serum-containing base medium supplemented with FGF2 alone as a growth factor.

37. The graft material for treating nerve damage according to claim 35, wherein the medium substantially containing no growth factors except FGF2 is a commercially available medium for culturing mesenchymal stem cells supplemented with FGF2 alone as a growth factor.

38. The graft material for treating nerve damage according to claim 35, wherein the nerve damage is spinal cord injury, cerebral infarction, intracerebral hemorrhage, subarachnoid hemorrhage, spinal hemorrhage, compression injury of nerve caused by disk herniation, sciatic nerve pain or peripheral nerve damage caused by diabetes.

39. A method for treating nerve damage, comprising
a step of grafting a graft material for treating nerve damage produced by the method according to claim 28 to an area of nerve damage.

40. The method according to claim 39, wherein the nerve damage is spinal cord injury, cerebral infarction, intracerebral hemorrhage, subarachnoid hemorrhage, spinal hemorrhage, compression injury of nerve caused by disk herniation, sciatic nerve pain or peripheral nerve damage caused by diabetes.

41. A kit for producing a graft material for treating nerve damage according to claim 35.

42. A method for selecting a material for a graft material for treating nerve damage from a plurality of groups of dental pulp stem cells, comprising selecting a dental pulp stem cell having at least one of the following properties (i) to (iv):
(i) the expression level of 10% or more of genes in the group of genes listed in Table 1 is 5 times or more as high as other groups of cells,
(ii) the expression level of at least one gene selected from the group consisting of MYO1G, RBMY2FP, FILIP1, C1orf64, TNFRSF8, C2orf48, AGTR1, Dydc2, Znf708, Dct, Slc15a1, Zhddc22, Adam20, Gnao1, Csn2, Semg2, Dnah1, Ctag1a, Lrrc19, Lipg and Cbln2 is 5 times or more as high as other groups of cells,
(iii) the expression level of 10% or more of genes in the group of genes listed in Table 2 is 5 times or more as low as other groups of cells, and
(iv) the expression level of at least one gene selected from the group consisting of Gafa3, Lmf1, Fam13a, Gkn1, Gpr112, Sult1c4, Slc35f4, Gstt1, Gpr27, Pdia2, RIIAD1, GSTT1, SLC2A2, and HTATSF1P2 is 5 times or more as low as other groups of cells.
